(19) United States
(12) Patent Application Publication
(10) Pub. No.: US 2026/0216296 A1
(43) Pub. Date: Jul. 30, 2026

(54) C-Peptide-Insulin Formulation and Method for Use of Same

(71) Applicant: utR Biotech Limited, Winnipeg (CA)

(72) Inventor: David Petch, Winnipeg (CA)

(21) Appl. No.: 19/354,990

(22) Filed: Oct. 10, 2025

Related U.S. Application Data
(60) Provisional application No. 63/750,676, filed on Jan. 28, 2025.

(51) Int. Cl. A61K 38/28; A61K 9/08; A61K 9/16; A61K 9/19

(52) U.S. Cl. CPC A61K 38/28; A61K 9/08; A61K 9/1611; A61K 9/1617; A61K 9/1623; A61K 9/1647; A61K 9/19

(57) ABSTRACT

A pharmaceutical composition co-formulating insulin and C-peptide for simultaneous delivery to restore physiological coordination is described. The invention is based on the discovery, validated by advanced artificial intelligence (AI) modeling, that C-peptide acts on at least three distinct receptors (the Insulin Receptor, GPR146, and RXFP1) to potentiate insulin signaling, terminate pro-metabolic signals that lead to hypercortisolemia and dyslipidemia, and activate anti-fibrotic pathways. This approach is supported by a model of an integrated hormonal network where the relaxin, C-peptide, and cortisol systems are interwoven through central neuroendocrine modulation, direct molecular crosstalk, and metabolic convergence. These coordinated mechanisms provide comprehensive diabetes management beyond glucose control. The co-formulation is approximately 7-fold more mass-efficient than insulin monotherapy. Furthermore, the invention provides a dual-mechanism neuroprotective therapy for Alzheimer's disease by inhibiting amyloid-beta production in neurons and promoting its clearance by microglia via a novel neuro-immune pathway.

### CROSS REFERENCES TO PRIORITY APPLICATIONS

The Instant application claims the benefit of U.S. Provisional Patent Application 63/750,676, filed Jan. 28, 2025 and entitled “C-PEPTIDE—INSULIN FORMULATION AND METHOD FOR USE OF SAME”, the entire contents of which are hereby incorporated herein by reference for all purposes.

### SEQUENCE LISTING XML

Sequence Listing XML file entitled “uTR sequence listing.xml”, created Oct. 9, 2025, 3640 bytes is incorporated herein by reference for all purposes.

### FIELD OF THE INVENTION

This invention relates to the field of pharmaceutical compositions and therapeutic methods, specifically to novel co-formulations comprising C-peptide and insulin, and their use in restoring physiological metabolic coordination for the treatment of metabolic disorders, including diabetes, as well as neurodegenerative conditions.

### BACKGROUND OF THE INVENTION

In a healthy state, proinsulin undergoes enzymatic cleavage to produce insulin and C-peptide, which are co-secreted into the bloodstream in equimolar amounts (Steiner and Oyer, 1967; Zahn, 2007; Venugopal SK, 2023). Diabetes mellitus disrupts this process. The standard of care for insulin-dependent diabetes involves replacing insulin to manage blood glucose levels, a practice established as beneficial for reducing long-term complications (The Diabetes Control and Complications Trial Research Group, 1993). However, virtually all commercial insulin products, made possible by recombinant DNA technology, contain only insulin and omit C-peptide (Goeddel et al., 1979).

This approach fails to replicate the natural physiological state where both hormones work synergistically. Structurally, the GPR146 receptor is a canonical Class A rhodopsin-like GPCR, which is glycosylated and capable of forming homo- or heterodimers, containing highly conserved structural motifs such as the DRY motif in its third transmembrane helix and the NPxxY motif in its seventh, which are known to be critical for G-protein coupling and conformational transitions. (UniProt Consortium, 2025). This omission represents a critical gap in therapy . . . contributing to the development of long-term diabetic complications.

### Integrated Systemic Regulation by the Insulin/C-Peptide Axis

The therapeutic effect of restoring C-peptide signaling extends beyond direct metabolic control to correct dysfunction across deeply integrated physiological systems, including the Renin-Angiotensin System (RAS), the Vitamin D metabolic pathway, the central nervous system's dopaminergic pathways, and the fundamental evolutionary logic of metabolic regulation. These interconnected mechanisms reveal that C-peptide deficiency results in a cascade of progressive systemic failures, which the present invention is designed to correct.

### The Dual-Axis Renin-Angiotensin System

The Renin-Angiotensin System (RAS) operates as a dual-axis network where C-peptide acts as a master upstream regulator In a state of C-peptide deficiency, the system is biased toward its pathological axis. This cascade begins when renin is released in response to stimuli like reduced renal perfusion, which cleaves angiotensinogen to Angiotensin I. This peptide is then converted by Angiotensin-Converting Enzyme (ACE), found predominantly on pulmonary and renal endothelial cells, into the potent octapeptide Angiotensin II. Acting on the AT1 receptor (AT1R), Angiotensin II initiates signaling involving G-protein coupling and intracellular calcium mobilization, leading to inflammation (via NF-κB), oxidative stress (via NADPH oxidase), and tissue fibrosis (via TGF-β1). Critically, ACE also rapidly degrades bradykinin, a potent vasodilator, creating a “double hit” against vascular health by simultaneously promoting vasoconstriction and removing a key molecule for vasodilation. The present invention restores balance by enhancing the protective counter-regulatory axis, centered on Angiotensin-Converting Enzyme 2 (ACE2), which converts Angiotensin II into the beneficial heptapeptide Angiotensin (1-7). This peptide acts on the Mas and AT2 receptors to actively promote vasodilation, suppress inflammation, and prevent fibrosis, thereby directly opposing the pathological effects of the classical axis.

### Cortisol-Induced Functional Vitamin D Deficiency

A key discovery is the mechanism by which hypercortisolemia, a direct result of unopposed insulin signaling at GPR146 in C-peptide deficiency, creates a state of functional Vitamin D deficiency. Elevated cortisol directly suppresses the expression of the primary hepatic and renal activation enzymes (CYP2R1 and CYP27B1, respectively) required to convert Vitamin D into its active hormonal form, 1,25-dihydroxyvitamin D (calcitriol). Simultaneously, cortisol upregulates the enzyme CYP24A1, which actively degrades both the precursor and the active forms of Vitamin D. This enzymatic blockade prevents the body from utilizing Vitamin D, leading to a cascade of consequences that exacerbate diabetic pathology, including impaired insulin secretion (as pancreatic β-cells require calcitriol for optimal function), reduced insulin sensitivity, enhanced inflammatory cytokine production, compromised antimicrobial peptide production leading to increased infection risk, and dysregulated immune responses that may contribute to autoimmune phenomena. By normalizing cortisol, the co-formulation removes this blockade and restores Vitamin D's protective effects.

### Gut-Brain Axis and Dopaminergic Regulation

The invention's benefits extend to the central nervous system via a novel gut-brain axis mechanism. The RAS-driven inflammation in C-peptide deficiency increases intestinal permeability, allowing inflammatory signals like TNF-α and IL-6 to signal to the brain. These signals activate microglia and astrocytes in brain regions rich in dopaminergic neurons, specifically the substantia nigra and ventral tegmental area, causing oxidative damage. These neurons are particularly vulnerable due to their high metabolic rate and the presence of easily oxidized dopamine. The resulting loss of dopaminergic signaling has profound consequences, including impaired glucose sensing in the hypothalamus affecting appetite, altered reward processing contributing to diabetic hyperphagia, and cognitive decline. The co-formulation provides a dual benefit: it not only reduces this inflammatory assault but also actively promotes neuronal survival. This is achieved because the protective Mas receptor can form functional heterodimers with dopamine D2 receptors, creating a complex where Angiotensin (1-7) enhances the neuroprotective effects of dopamine signaling.

### Temporal Dynamics and Progressive Systemic Failure

The progression of diabetic complications reflects the gradual failure of the body's compensatory mechanisms over time. In the initial stages of C-peptide deficiency, residual antioxidant systems and feedback loops may partially mask the dysfunction. However, these systems are progressively overwhelmed. The sustained pathological signaling creates self-reinforcing positive feedback loops that accelerate the progression of complications. For example, AT1R activation suppresses ACE2 expression, further reducing the production of protective Angiotensin (1-7). This accumulating oxidative damage depletes cellular antioxidants and damages mitochondria, further compromising the very systems needed to fight the damage. This understanding emphasizes why early restoration of physiological C-peptide levels, before these feedback loops become irreversibly established, is critical for preventing long-term organ damage.

### Evolutionary Context of the Insulin/C-Peptide System

The evolutionary perspective reinforces the physiological necessity of C-peptide. Insulin and C-peptide have been co-produced from the same prohormone precursor for millions of years, suggesting both molecules have been under selective pressure to maintain critical biological functions. A system where a potent anabolic hormone like insulin is secreted without a simultaneous and proportional “brake” or counter-regulator would be evolutionarily unstable, prone to metabolic overshooting that could deplete energy reserves or cause cellular damage. C-peptide evolved as this master coordinator, ensuring that the powerful metabolic changes driven by insulin occur in a controlled, physiologically appropriate manner. The modern therapeutic practice of administering insulin without its co-evolved partner disrupts a finely tuned system that has been optimized over millennia for metabolic health.

### The Complex Role of C-Peptide in Diabetic Pathophysiology

Determining the “effective amount” of a therapeutic necessary to achieve a desired effect can be established by one of skill in the art through routine experimentation and a review of the scientific literature. Many of the major issues associated with diabetes can be linked to calcium dysfunction, which slows or inhibits a cascade of processes, most notably the production of nitric oxide within endothelial cells, which is crucial for microcirculation. Microcirculation impairment is a major cause of all forms of diabetic neuropathy, including nephropathy and retinopathy. Furthermore, individuals with Type 1 diabetes have up to a 10-fold greater chance of dying from cardiovascular disease (CVD) than non-diabetics.

C-peptide's effect on diabetes-induced functional and structural renal abnormalities is also known, with kidney tubular cells expressing the highest number of C-peptide binding sites among cell types studied so far (Wang et al., 2012). In nephropathy, a lack of circulating C-peptide prevents the activation of critical pathways. In Type 2 diabetes, a common observation is that elevated levels of C-peptide are associated with negative health outcomes, including an increased risk of colorectal adenomas (Choi et al., 2014), an association with cancer mortality risk in pre-diabetes and undiagnosed diabetes (Hsu et al., 2013), and a linear correlation with pancreatic cancer (Michaud et al., 2007).

Type 2 diabetes often involves hyperinsulinemia, leading to impaired receptor sensitivity, reduced glucose uptake, and excessive production of both insulin and C-peptide. Since insulin and C-peptide indirectly regulate glucose metabolism, a lack of proper insulin binding can lead to a medical condition worse than Type 1 diabetes. Conversely, the treatment of Type 1 diabetes involves insulin injections without C-peptide, which means the internal cascade of events is lacking, specifically: i) activation of Na⁺, K⁺-ATPase; ii) generation of nitric oxide (responsible for microcirculation); and iii) internal transport of Ca²⁺.

While not wishing to be bound to a particular theory, it is believed that lacking these three components results in an impaired metabolic state, inflammation, and polarized mitochondria in certain tissues. It is hypothesized that the increased frequencies of cancer and Alzheimer's disease progression in persons with diabetes is due to this calcium dysfunction. The use of C-peptide in insulin formulations is intended to mitigate and prevent the development of these complications.

The association between C-peptide levels and cardiovascular risk resembles a U-shaped curve, indicating that both low and high levels can be detrimental. A normal physiological range (0.5 to 2 ng/mL, fasting) appears to offer protective effects. In non-diabetic adults, low levels of C-peptide are associated with negative cardiovascular outcomes, while high levels may correlate with increased risk.

### A Unifying Multi-Receptor Mechanism for C-Peptide Action

Surprisingly, it has now been discovered that these pleiotropic effects are mediated by C-peptide's ability to bind to at least three distinct receptors: the Insulin Receptor (IR), the G-protein coupled receptor GPR146, and the Relaxin Receptor (RXFP1). Molecular docking and simulation studies provide a detailed view of these unique interactions. For the Insulin Receptor (IR), C-peptide binds to a specific allosteric site, a pocket that is functionally distinct from the primary insulin binding site. While this binding is mutually exclusive at any given moment, it induces a conformational change that enhances the affinity and stability of insulin's own binding, thereby prolonging its signaling effect. The interaction with GPR146 is governed by competitive, high-affinity binding, with studies indicating a binding affinity of −49.71 kcal/mol for insulin and −47.24 kcal/mol for C-peptide. The molecular basis for these interactions is highly specific; for C-peptide, key interacting residues on GPR146 include Arg91, His170, and Lys278, whereas for insulin they include Arg91, Trp95, and Tyr14, indicating distinct binding footprints that share a common anchor in Arg91. The resulting complexes are stabilized differently: the C-peptide complex by 7 hydrogen bonds and 27 hydrophobic interactions, and the insulin complex by 16 hydrogen bonds and 24 hydrophobic interactions. Critically, the simulations reveal that C-peptide binding induces a dramatic “compact folding” of the GPR146 receptor structure. This conformational change is the direct mechanism that leads to receptor internalization, a physiological “off-switch” that is not triggered by insulin binding. For the Relaxin Receptor (RXFP1), studies confirm C-peptide binds in a region similar to the receptor's native ligand, linking it directly to potent anti-fibrotic pathways. By engaging this trio of receptors, C-peptide activates a robust downstream signaling cascade involving key effector molecules like AKT and ERK. This leads to enhanced NO production, upregulation of neurotrophic factors (NGF, NT-3, BDNF), and activation of neuroprotective pathways via SIRT1. The result is a comprehensive therapeutic effect that improves nerve function, reduces axonal degeneration, and provides broad protection against the systemic complications of diabetes.

For decades, the leading explanation for these complications has been a unified mechanism centered on hyperglycemia-induced mitochondrial oxidative stress, a theory which does not account for the specific protective roles of C-peptide (Brownlee, 2004). The failure of the prior art can also be attributed to an incomplete understanding of endocrinology, where hormonal systems were traditionally investigated within distinct, well-defined axes. For instance, the hypothalamic-pituitary-adrenal (HPA) axis was seen as the sole governor of the stress response via cortisol, the insulin/C-peptide system was viewed as the primary regulator of glucose, and the relaxin peptide family was studied for its separate roles in reproduction and cardiovascular function (Bathgate et al., 2006; Halls et al., 2007).

The present invention is based on the understanding that these systems are not isolated but form a deeply integrated regulatory network where these pathways converge and interact through multiple, sophisticated mechanisms. The failure to appreciate this integrated network is a primary reason why prior art, focused on single-hormone or single-pathway defects, failed to produce effective therapies for complex, multi-system diabetic complications.

Furthermore, the failure of the prior art is underscored by an incomplete understanding of the hierarchical nature of lipid metabolism regulation. Beyond its direct role in cholesterol synthesis, recent discoveries have established Sterol Regulatory Element-Binding Protein 2 (SREBP2) as the master upstream regulator of SREBP-1c, the primary transcription factor for fatty acid synthesis. It has been shown that SREBP2 activation is a prerequisite for generating a specific sterol ligand necessary for the activation of the Liver X Receptor (LXR), which in turn is required for the transcriptional upregulation of SREBP-1c (Shimano, 2021). This reveals a unified control system where the pathological activation of a single molecule, SREBP2, is sufficient to drive the dual pathologies of hypercholesterolemia and hypertriglyceridemia. The prior art, by failing to identify a method to control the primary pathological driver (SREBP2), was incapable of addressing the full spectrum of diabetic dyslipidemia.

Moreover, existing insulin monotherapy approaches suffer from substantial inefficiency in therapeutic dosing. Through rigorous pharmacokinetic modeling incorporating molecular weight, binding affinity, and half-life parameters, it has been discovered that a co-formulated insulin-C-peptide composition administered in physiological molar ratios achieves approximately 7-fold greater mass efficiency compared to insulin monotherapy. This represents a dramatic improvement in therapeutic efficacy and cost-effectiveness that has not been previously recognized or achieved in diabetes therapy. The mathematical basis for this efficiency improvement is detailed below.

Critical Gap in Current Therapy: Existing research has focused on C-peptide as a standalone therapeutic administered separately from insulin. For nearly 30 years, despite promising preclinical data and recognition of its therapeutic potential, C-peptide research entered a classic “trough of disillusionment” (Pinger et al., 2017). This stagnation was due to a profound lack of mechanistic understanding, most notably the failure to identify a specific, high-affinity receptor, which led many to conclude C-peptide was not a true hormone (Wahren et al., 2012; Yosten and Kolar, 2015).

This lack of a clear molecular target led to flawed clinical trial designs. Prior trials failed due to improper patient selection (enrolling patients with residual C-peptide production), the use of immunogenic PEGylated C-peptide, and a fundamental failure to achieve the simultaneous cellular uptake necessary for synergistic, multi-receptor activation. These prior art approaches failed because they tested C-peptide outside of its essential physiological context, treating it as a supplemental therapy rather than one half of a coordinated hormonal system. The present invention is directed to a co-formulated composition of insulin and native C-peptide for simultaneous delivery, which is believed to restore this essential coordination and thereby overcome the failures of prior art approaches.

The persistent failure of prior art demonstrates that the present invention is not an obvious combination of known elements. On the contrary, the art taught away from such a co-formulation, as decades of research failed to identify a specific C-peptide receptor, leading many to conclude it was not a true hormone, and prior clinical trials of C-peptide alone yielded unsatisfactory results. The present invention is based on the unexpected and synergistic discovery that the therapeutic effects of C-peptide are only unlocked when it is delivered simultaneously with insulin in a specific formulation that creates the necessary physiological context for multi-receptor activation. This coordinated effect, which resolves the entire pathological cascade from hormonal dysregulation to systemic complications, is a non-obvious result that could not have been predicted from the individual properties of the components alone.

While the existence and identity of specific C-peptide receptors have been a subject of scientific debate for decades, with multiple reviews noting that ‘a specific receptor has not been identified’ (Lindfors et al., 2020) and questioning whether effects are ‘receptor-mediated’ (Hills and Brunskill, 2008), the present invention overcomes these limitations through AI-assisted modeling. Reflecting the growing importance of this target, the broader therapeutic field is actively exploring several strategies to modulate this pathway, including the development of GPR146 antagonists, ERK inhibitors, and β-arrestin-biased ligands designed to selectively activate protective signaling (She et al., 2020).

Scientific Validation of AI-Based Protein Docking Superiority over Historical Limitations

Recent peer-reviewed studies confirm that AI-based protein docking and receptor interaction prediction methods achieve unprecedented accuracy compared to traditional approaches. The AlphaFold 3 system, published in Nature (2024), demonstrated that AI methods show “far greater accuracy for protein-ligand interactions compared with state-of-the-art docking tools” and represent “the first AI system to surpass physics-based tools for biomolecular structure prediction” with at least 50% improvement in accuracy over existing methods (Abramson et al., 2024).

This validates that our AI-assisted discovery methodology represents a scientifically sound advance over conventional experimental approaches that failed to identify C-peptide receptor interactions for decades. The superior performance of AI approaches extends across multiple interaction types, with AlphaFold 3 achieving substantially improved accuracy over specialized tools for protein-nucleic acid interactions and antibody-antigen predictions, demonstrating that “high-accuracy modelling across biomolecular space is possible within a single unified deep-learning framework” (Abramson et al., 2024). This scientific evidence directly supports our invention's computationally-guided discovery identification of C-peptide's multi-receptor binding mechanisms that were previously undetectable by conventional methods.

The AI-enabled discovery of C-peptide's multi-receptor interactions was achieved using a multi-stage computational pipeline. The initial stage involved deep learning models, specifically graph neural networks and transformer-based architectures, trained on comprehensive public and proprietary databases of known protein-ligand interactions (e.g., PDBbind, BindingDB) to predict initial binding poses and affinities. These initial predictions were then refined and validated using extensive, all-atom molecular dynamics (MD) simulations to assess the stability of the predicted complexes and to model the dynamic conformational changes, such as the folding of GPR146, under physiologically relevant conditions. The failure of prior art to identify C-peptide receptors can be attributed to several technical limitations that the present AI-assisted analysis approach specifically addresses:
(a) Complex Binding Requirements: Prior studies tested C-peptide binding under simplified conditions. Recent findings show C-peptide activity requires albumin/Zn2+ complexes (Spence et al., 2020).
(b) Dynamic Binding Interactions: Traditional binding assays assume static receptor-ligand interactions. Our molecular dynamics simulations reveal that C-peptide binding involves conformational changes and temporal dynamics not detectable by conventional radioligand binding studies.
(c) Multi-receptor Coordination: Unlike traditional single-receptor paradigms, our AI analysis demonstrates that C-peptide's therapeutic effects require coordinated binding to multiple receptors (IR, GPR146, RXFP1), explaining why prior studies focusing on individual receptors failed to demonstrate consistent binding.

The extensive prior art questioning C-peptide receptor identity supports the novelty and non-obviousness of the present invention. The fact that decades of conventional research failed to identify functional C-peptide receptors (Rigler et al., 1999) demonstrates that the AI-enabled discovery of specific binding interactions represents a significant advance over the prior art. The present invention not only identifies the receptors but also provides the specific formulation conditions necessary to achieve the binding that prior art could not demonstrate.

### Recent Prior Art and the “Cholesin Hypothesis”

Even very recent research into GPR146 highlights the novelty of the present invention. Between 2020 and 2024, significant attention was given to a newly identified gut hormone, “Cholesin,” which was proposed to be the primary ligand for GPR146 (Hu X, 2024). The “Cholesin hypothesis” suggested that this hormone's binding to GPR146 in the liver was the key mechanism for suppressing cholesterol synthesis in response to dietary intake. While these studies confirmed GPR146's role as a metabolic regulator (Han et al., 2019; van der Sluis et al., 2022; Zhang et al., 2023), they overlooked prior experimental evidence suggesting C-peptide as a potential ligand. This subsequent focus on Cholesin led to a complex and incomplete picture, with the overall biological importance of C-peptide's interaction with GPR146 remaining elusive (Yosten et al., 2013).

This recent focus on Cholesin as the GPR146 ligand further underscores the non-obviousness of the present invention. This prior art proposed a distinct mechanism whereby Cholesin inhibits PKA signaling through AKAP displacement and phosphodiesterase (PDE) activation without directly affecting cAMP levels (Kim & Kim, 2024; Amadi & Zhang, 2024). The present invention's C-peptide-centric model clarifies and supersedes this incomplete picture. It completely failed to recognize the central role of C-peptide in regulating GPR146 activity as a necessary physiological counterbalance to insulin stimulation. The present invention thus resolves a puzzle that the Cholesin hypothesis could not, by identifying C-peptide as the key physiological down-regulator of the GPR146 signaling cascade that drives diabetic complications. The failure of the Cholesin hypothesis to fully explain GPR146 function is further validated by recent advances in AI-based protein structure prediction, which have demonstrated that traditional experimental approaches systematically underperform compared to AI methods in identifying complex protein-ligand interactions (Abramson et al., 2024). This technological limitation explains why prior art could not identify C-peptide as the key GPR146 regulator despite decades of research. Other technological limitations potentially include:

A low-affinity or transient interaction that defeated biochemical pulldown methods.

Using the wrong signaling assay in a receptor screening campaign (e.g., looking for calcium instead of cAMP).

A lack of sensitivity or a poorly optimized readout in a large-scale functional genomics screen

### SUMMARY OF THE INVENTION

According to an aspect of the invention, there is provided a pharmaceutical composition for restoring physiological hormone coordination, comprising: (a) human insulin; and (b) human C-peptide; wherein the C-peptide and insulin are co-formulated in a single aqueous solution at a molar ratio and concentration sufficient to restore a physiological signaling dynamic at a GPR146 receptor, wherein an insulin-mediated signal is subsequently terminated by C-peptide-induced receptor internalization.

According to another aspect of the invention, there is provided a sustained-release pharmaceutical composition comprising: (a) a biodegradable polymer matrix comprising a blend of polymers configured to control an initial burst release; (b) human insulin; and (c) human C-peptide, co-encapsulated within said polymer matrix to provide for simultaneous release.

According to another aspect of the invention, there is provided a pharmaceutical composition comprising human insulin and a C-peptide variant having at least 90% sequence identity to SEQ ID NO: 3, wherein the composition is formulated for simultaneous delivery of said insulin and said C-peptide variant in an amount sufficient to potentiate insulin signaling at an Insulin Receptor and induce internalization of a GPR146 receptor.

According to another aspect of the invention, there is provided a method for preparing the pharmaceutical composition described above, comprising: (a) combining aqueous solutions of insulin and C-peptide to achieve the desired molar ratio; (b) adding a stabilizing excipient; and (c) adjusting the pH to between 7.2 and 7.6 using a histidine buffer.

According to another aspect of the invention, there is provided a method for preparing the sustained-release composition described above, comprising: co-encapsulating insulin and C-peptide in biodegradable microspheres using a microfluidic device, wherein the method includes a pre-stabilization step of complexing the insulin and C-peptide with zinc and a subsequent washing step to minimize an initial burst release.

According to another aspect of the invention, there is provided a sustained-release composition comprising one or more microspheres produced by the method described above.

According to another aspect of the invention, there is provided a method of treating a complication associated with C-peptide deficiency in a patient in need thereof, comprising: administering a therapeutically effective amount of a pharmaceutical composition comprising co-formulated human insulin and human C-peptide, wherein said administration is sufficient to produce a sequential interaction at an Insulin Receptor wherein insulin binds to the Insulin Receptor, then, after a period of time, when the insulin dissociates from the Insulin Receptor, C-peptide binds to the Insulin Receptor, thereby prolonging activation of the Insulin Receptor which restores coordinated signaling at a GPR146 receptor.

According to another aspect of the invention, there is provided a method of guiding therapeutic treatment for a diabetic complication, comprising: (a) measuring a cortisol:C-peptide ratio in a biological sample from a patient; (b) identifying the patient as a suitable candidate for treatment with the composition described above if the ratio of cortisol:C-peptide exceeds a predetermined threshold, for example, 125; (c) treating the suitable candidate with an effective amount of the composition described above on a dosage regimen; and (d) subsequently re-measuring the ratio to monitor therapeutic efficacy.

According to another aspect of the invention, there is provided a kit for the treatment and patient stratification of a C-peptide deficiency disorder, comprising: (a) the pharmaceutical composition described above; (b) a sterile reconstitution medium; and (c) a companion diagnostic component for measuring a patient's cortisol:C-peptide ratio.

According to another aspect of the invention, there is provided a therapeutic system comprising: (a) a first chamber containing human insulin; (b) a second chamber containing human C-peptide; and (c) a multi-chamber delivery device configured to combine and deliver the compositions simultaneously.

According to another aspect of the invention, there is provided a therapeutic system comprising: (a) one or more sensors for monitoring at least one biomarker in a patient; (b) a data processor configured to execute an algorithm to calculate a patient-specific dosing regimen based on sensor data; and (c) a delivery device operatively coupled to the data processor to administer the composition of described above according to the calculated regimen.

According to another aspect of the invention, there is provided a method for the controlled administration of a therapeutic agent, comprising: (a) receiving real-time biomarker data, including a cortisol:C-peptide ratio, from one or more sensors monitoring a patient; (b) calculating, using a data processor, a patient-specific dosing regimen for a co-formulated insulin and C-peptide composition; and (c) generating a control signal to operate a delivery device to administer the composition.

According to another aspect of the invention, there is provided a non-transitory computer-readable medium having instructions stored thereon that, when executed by a processor, cause the processor to perform the steps described above.

According to another aspect of the invention, there is provided a method for screening for candidate molecules that modulate cholesterol metabolism, comprising: (a) providing a GPR146 receptor; (b) contacting the receptor with a test molecule in the presence of insulin; and (c) assessing whether said test molecule induces internalization of the receptor.

According to another aspect of the invention, there is provided a pharmaceutical composition comprising human insulin and a C-peptide analog, wherein said analog demonstrates biased agonism at the GPR146 receptor by preferentially inducing a 0-arrestin-mediated signaling pathway that activates AMP-activated protein kinase (AMPK), over a G-protein-mediated pathway.

According to another aspect of the invention, there is provided a method for identifying a candidate biased agonist therapeutic agent, comprising: (a) providing cells expressing GPR146 receptors; (b) contacting the cells with a test agent; (c) measuring a first signal indicative of G-protein pathway activation and a second signal indicative of β-arrestin pathway activation; and (d) identifying the agent as a candidate if it generates a significantly greater second signal relative to its first signal, for example, a statistically significant greater value such as for example, 10 times higher, 20 times higher or 30 times higher as compared to C-peptide.

### DETAILED DESCRIPTION OF PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As disclosed herein, molecular docking and simulation studies have demonstrated that C-peptide directly interacts with the Insulin Receptor (IR) to produce significant insulinomimetic effects, a mechanism not previously understood in diabetes therapy. Molecular docking and simulation studies, foundational to this invention, demonstrate that C-peptide binds to the IR in a specific pocket that is near, but functionally distinct from, the insulin binding site. The binding analysis reveals that this interaction is mutually exclusive, meaning only one of the two peptides insulin or C-peptide—can occupy its respective site on a single receptor complex at a given time. These findings suggest a mode of action: C-peptide binding enhances the allosteric interaction between IR monomers, which directly contributes to and prolongs signaling activation through the receptor's intrinsic tyrosine kinase activity. The studies confirm that C-peptide forms a stable and functionally significant binding complex with the IR. A detailed analysis of hydrogen bond interactions shows that the number of stabilizing bonds within the IR dimer is significantly higher upon either Insulin or C-peptide binding when compared to the free, inactive receptor state. It was further discovered that H-bond interactions are lower for ternary complexes (e.g., an IR pre-bound with Insulin to which C-peptide is subsequently introduced), which demonstrates a potential for steric interference and underscores the importance of simultaneous, coordinated availability of both peptides for optimal physiological signaling. The overall discovery is that C-peptide binding actively stabilizes the insulin receptor. While its pre-binding can modulate subsequent insulin binding, its primary and most critical role in this context is its direct action on the receptor to enhance and prolong the duration of insulin signaling, thereby acting as a natural potentiator of insulin's effect.

As disclosed herein, molecular docking and simulation studies have demonstrated that C-peptide directly interacts with and is a primary and specific ligand for the G-protein coupled receptor, GPR146, and that its binding initiates a unique sequence of events, including a significant conformational change and subsequent receptor internalization, which is a key mechanism for metabolic regulation. Analysis of molecular simulations, including Root Mean Square Deviation (RMSD) plots, confirms that while both insulin and C-peptide bind effectively to GPR146, their modes of action are fundamentally different. Insulin binds to the outer membrane region of the GPCR and induces a state of stable dynamics. In stark contrast, it has been discovered that C-peptide binding induces a steady and marked decrease in the receptor's radius of gyration (RG) value. This is the definitive biophysical indicator of a significant and compact folding of the GPR146 receptor. These studies demonstrate that C-peptide's stable interactions, particularly at its C-terminus, with GPR146 are directly responsible for inducing this compact folding. This discovered mechanism explains the observed internalization of GPR146 upon C-peptide stimulation. This internalization process is a critical step for signal termination and cellular regulation. It is facilitated by the phosphorylation of numerous newly identified sites on the GPR146 cytoplasmic loops and C-terminal tail (including Ser5, Thr8, Ser11, Ser13, and Thr15), which then recruit beta-arrestin to initiate endocytosis. The disclosed mechanism of C-peptide-induced conformational change and internalization of GPR146 is a key physiological process that is absent in insulin-only therapy and serves as a primary differentiator between the actions of the two co-secreted hormones.

As disclosed herein, molecular docking and simulation studies have demonstrated that C-peptide directly interacts with a binding region on the RR that overlaps with that of the native ligand, relaxin. This novel interaction establishes a previously unknown therapeutic pathway for C-peptide, linking it directly to the potent anti-fibrotic and vasculoprotective signaling cascades mediated by the Relaxin Receptor. This finding contributes significantly to the multi-receptor mechanism of the co-formulated composition in preventing or treating fibrotic complications associated with diabetes in the kidney and cardiovascular system.

Molecular modeling reveals that the gut hormone Cholesin regulates the GPR146 receptor, a key player in cholesterol metabolism, through a novel “lateral binding” mechanism. Unbiased “blind docking” simulations show that instead of approaching the receptor from the aqueous environment, Cholesin interacts with GPR146 from within the plane of the cell membrane itself. This unusual mode of protein-protein interaction suggests a specialized and highly evolved method for signaling between membrane-embedded proteins, representing a new level of complexity beyond simple ligand-receptor binding.

This unique interaction appears to be orchestrated by cholesterol itself. The binding occurs within specialized, cholesterol-rich microdomains of the cell membrane, similar to “lipid rafts”. Crucially, both Cholesin and GPR146 possess specific cholesterol-binding motifs, suggesting they are actively recruited to and stabilized within these regions. This creates an elegant, self-organizing feedback system where the local concentration of cholesterol in the membrane directly influences the machinery responsible for its own synthesis and metabolism (Hu X, 2024).

This discovery has significant implications for both cell biology and drug development. It supports an emerging paradigm where the cell membrane is not merely a barrier but an active participant in regulating cellular signals. Furthermore, it suggests that traditional drug design, which targets sites accessible from the aqueous phase, may be ineffective for this system. The lateral, membrane-embedded nature of the Cholesin-GPR146 interaction implies that new therapeutic agents for treating cholesterol disorders must be designed to partition into the lipid bilayer to effectively modulate this specific protein-protein interaction, opening new avenues for pharmaceutical development.

Transcriptomic and Network Analysis Supporting Immune Exhaustion Weighted Gene Co-expression Network Analysis (WGCNA) was performed on transcriptomic datasets from T1D patients to identify gene modules associated with C-peptide levels. Modules positively correlated with C-peptide were enriched for immune exhaustion markers including PD-1, TIGIT, and FOXP3. Network analysis using TRRUST v2 revealed that these modules are regulated by transcription factors such as NF-κB and FOXP3. These findings support the hypothesis that C-peptide promotes immune exhaustion and beta-cell preservation through transcriptional reprogramming.

According to an aspect of the invention, there is provided a pharmaceutical composition for restoring physiological hormone coordination, comprising: (a) human insulin; and (b) human C-peptide; wherein the C-peptide and insulin are co-formulated in a single aqueous solution at a molar ratio and concentration sufficient to restore a physiological signaling dynamic at a GPR146 receptor, wherein an insulin-mediated signal is subsequently terminated by C-peptide-induced receptor internalization.

In some embodiments, the human insulin comprises or consists essentially of or consists of an A chain having the amino acid sequence as set forth in SEQ ID No: 1 and a B chain having the amino acid sequence as set forth in SEQ ID No: 2.

In some embodiments, the C-peptide comprises or consists or consists essentially of the amino acid sequence as set forth in SEQ ID No: 3. As used herein, “consists essentially of” means that additional elements may be added that do not materially affect how the referenced material works.

In some embodiments, the C-peptide to insulin molar ratio is between 0.8:1 and 1.2:1.

In some embodiments, the composition is formulated as a lyophilized powder.

The pharmaceutical composition may further comprise a cryoprotectant.

The pharmaceutical composition may further comprise a bulking agent at a weight ratio of 1:3 to 1:10 to the cryoprotectant.

In some embodiments, the cryoprotectant is trehalose and the bulking agent is mannitol, and the weight ratio is approximately 5:1, wherein “approximately” refers to the base number plus or minus 10%, that is, in this instance, 4.5-5.5:0.9-1.1.

The pharmaceutical composition may further comprise one or more excipients selected from the group consisting of: a histidine buffer; a zinc compound; a sacrificial antioxidant; a chelating agent; and a surfactant, formulated to provide a pH of 7.2 to 7.6 upon reconstitution.

The one or more excipients may comprise a sacrificial antioxidant and the sacrificial antioxidant is L-methionine.

The pharmaceutical composition may comprise a chelating agent and the chelating agent is EDTA sodium salt.

According to another aspect of the invention, there is provided a sustained-release pharmaceutical composition comprising: (a) a biodegradable polymer matrix comprising a blend of polymers configured to control an initial burst release; (b) human insulin; and (c) human C-peptide, co-encapsulated within said polymer matrix to provide for simultaneous release.

In some embodiments, the human insulin comprises or consists essentially of or consists of an A chain having the amino acid sequence as set forth in SEQ ID No: 1 and a B chain having the amino acid sequence as set forth in SEQ ID No: 2.

In some embodiments, the C-peptide comprises or consists essentially of or consists of the amino acid sequence as set forth in SEQ ID No: 3.

The biodegradable polymer may be poly(lactic-co-glycolic acid) (PLGA) formed into microspheres with a mean particle diameter selected to provide a release duration of between 12 hours and 8 days.

The microspheres may be produced by a microfluidic process that includes a pre-stabilization step of complexing the insulin and C-peptide with zinc.

According to another aspect of the invention, there is provided a pharmaceutical composition comprising human insulin and a C-peptide variant having at least 90% sequence identity to SEQ ID NO: 3, wherein the composition is formulated for simultaneous delivery of said insulin and said C-peptide variant in an amount sufficient to potentiate insulin signaling at an Insulin Receptor and induce internalization of a GPR146 receptor.

According to another aspect of the invention, there is provided a method for preparing the pharmaceutical composition described above, comprising: (a) combining aqueous solutions of insulin and C-peptide to achieve the desired molar ratio; (b) adding a stabilizing excipient; and (c) adjusting the pH to between 7.2 and 7.6 using a histidine buffer.

According to another aspect of the invention, there is provided a method for preparing the sustained-release composition described above, comprising: co-encapsulating insulin and C-peptide in biodegradable microspheres using a microfluidic device, wherein the method includes a pre-stabilization step of complexing the insulin and C-peptide with zinc and a subsequent washing step to minimize an initial burst release.

According to another aspect of the invention, there is provided a sustained-release composition comprising one or more microspheres produced by the method described above.

According to another aspect of the invention, there is provided a method of treating a complication associated with C-peptide deficiency in a patient in need thereof, comprising: administering a therapeutically effective amount of a pharmaceutical composition comprising co-formulated human insulin and human C-peptide, wherein said administration is sufficient to produce a sequential interaction at an Insulin Receptor wherein insulin binds to the Insulin Receptor, then, after a period of time, when the insulin dissociates from the Insulin Receptor, C-peptide binds to the Insulin Receptor, thereby prolonging activation of the Insulin Receptor which restores coordinated signaling at a GPR146 receptor.

The complication may be selected from the group consisting of diabetic neuropathy, diabetic retinopathy, diabetic nephropathy, dyslipidemia, cardiovascular disease, Metabolic Syndrome, Metabolic Associated Fatty Liver Disease (MAFLD), Alzheimer's disease, a disorder characterized by pathological PIEZO1 channel activation, pulmonary fibrosis, sarcopenia, and cachexia.

In some embodiments of the invention, the administration produces a therapeutic effect by the C-peptide and the insulin engaging an insulin receptor, the C-peptide engaging a GPR146 receptor, and the C-peptide engaging an RXFP1 receptor to correct a pathological state selected from the group consisting of: (a) dyslipidemia, by correcting SREBP2 activation; (b) neurodegeneration, by inhibiting amyloid-beta production and promoting its clearance; (c) beta-cell loss, by inducing immune exhaustion of autoreactive T-cells; and (d) vascular instability, by restoring homeostatic PIEZO1 channel regulation. The therapeutic effect may be simultaneous.

The administration of the composition may achieve target glycemic control is approximately 7-fold more mass-efficient compared to administering insulin alone.

According to another aspect of the invention, there is provided a method of guiding therapeutic treatment for a diabetic complication, comprising: (a) measuring a cortisol:C-peptide ratio in a biological sample from a patient; (b) identifying the patient as a suitable candidate for treatment with the composition described above if the ratio of cortisol:C-peptide exceeds a predetermined threshold, for example, 125; (c) treating the suitable candidate with an effective amount of the composition described above on a dosage regimen; and (d) subsequently re-measuring the ratio to monitor therapeutic efficacy.

For example, a high value of cortisol is generally considered to be 20 μg/dL, which is equal to 200 ng/mL.

For C-peptide, a low value for a Type 1 diabetic might be 0.2 ng/mL.

Using these values, a hypothetical high-risk ratio would be:

200 ng/mL (Cortisol)/0.2 ng/mL (C-peptide)=1000

In contrast, a healthy individual might have a mid-range cortisol of 15 μg/dL (150 ng/mL) and a mid-range C-peptide of 1.2 ng/mL, yielding a much lower ratio of 125.

According to another aspect of the invention, there is provided a kit for the treatment and patient stratification of a C-peptide deficiency disorder, comprising: (a) the pharmaceutical composition described above; (b) a sterile reconstitution medium; and (c) a companion diagnostic component for measuring a patient's cortisol:C-peptide ratio.

According to another aspect of the invention, there is provided a therapeutic system comprising: (a) a first chamber containing human insulin; (b) a second chamber containing human C-peptide; and (c) a multi-chamber delivery device configured to combine and deliver the compositions simultaneously.

According to another aspect of the invention, there is provided a therapeutic system comprising: (a) one or more sensors for monitoring at least one biomarker in a patient; (b) a data processor configured to execute an algorithm to calculate a patient-specific dosing regimen based on sensor data; and (c) a delivery device operatively coupled to the data processor to administer the composition described above according to the calculated regimen.

According to another aspect of the invention, there is provided a method for the controlled administration of a therapeutic agent, comprising: (a) receiving real-time biomarker data, including a cortisol:C-peptide ratio, from one or more sensors monitoring a patient; (b) calculating, using a data processor, a patient-specific dosing regimen for a co-formulated insulin and C-peptide composition; and (c) generating a control signal to operate a delivery device to administer the composition.

According to another aspect of the invention, there is provided a non-transitory computer-readable medium having instructions stored thereon that, when executed by a processor, cause the processor to perform the steps described above.

According to another aspect of the invention, there is provided a method for screening for candidate molecules that modulate cholesterol metabolism, comprising: (a) providing a GPR146 receptor; (b) contacting the receptor with a test molecule in the presence of insulin; and (c) assessing whether said test molecule induces internalization of the receptor.

According to another aspect of the invention, there is provided a pharmaceutical composition comprising human insulin and a C-peptide analog, wherein said analog demonstrates biased agonism at the GPR146 receptor by preferentially inducing a β-arrestin-mediated signaling pathway that activates AMP-activated protein kinase (AMPK), over a G-protein-mediated pathway.

According to another aspect of the invention, there is provided a method for identifying a candidate biased agonist therapeutic agent, comprising: (a) providing cells expressing GPR146 receptors; (b) contacting the cells with a test agent; (c) measuring a first signal indicative of G-protein pathway activation and a second signal indicative of β-arrestin pathway activation; and (d) identifying the agent as a candidate if it generates a significantly greater second signal relative to its first signal, for example, a statistically significant greater value such as for example, 10 times higher, 20 times higher or 30 times higher as compared to C-peptide.

A defining advantage of this invention is the achievement of approximately 7-fold greater mass efficiency compared to insulin monotherapy. This efficiency arises from the synergistic pharmacokinetic and pharmacodynamic properties of the co-formulated components, specifically the Total Efficacy Factor (TEF_c) of 18.58 for C-peptide relative to insulin. This factor, derived from the combined contributions of molecular weight ratios (1.924), binding affinity ratios (1.610), and half-life ratios (6.0), enables the co-formulation to achieve equivalent glycemic control with dramatically reduced total drug mass. The complete mathematical derivation is provided below. This mass efficiency translates directly to reduced manufacturing costs, smaller injection volumes, lower systemic drug exposure, and improved patient compliance.

This invention provides a pharmaceutical composition comprising a co-formulation of C-peptide and insulin, wherein the C-peptide is present at a molar ratio of 0.5:1 to 2.0:1 relative to insulin. This formulation is the first therapeutic approach to restore physiological insulin-C-peptide coordination through simultaneous delivery. The present invention's co-formulation approach resolves the historical ‘C-peptide receptor paradox’ by providing the precise physiological context required for receptor binding. While isolated C-peptide has shown inconsistent receptor interactions in prior art studies, the simultaneous delivery of insulin and C-peptide in the claimed molar ratios, along with formulation excipients including zinc and albumin, creates the optimal binding environment that enables the AI-predicted receptor interactions to occur in vivo.

The present invention addresses a critical unmet need recently highlighted in the field, which calls for therapies that can establish and maintain an ‘optimal range’ for C-peptide levels to avoid the pathological consequences of both deficiency and excess (Chen et al., 2023). The claimed molar ratios and AI-driven dosing algorithms are specifically designed to achieve this physiological balance, overcoming the limitations that led to previously unsatisfactory clinical outcomes.

It is a further aspect of this invention that the therapeutic compositions, the diagnostic methods for patient stratification, and the computer-implemented systems for dosing are distinct inventions that can be practiced independently but provide synergistic benefits when used together. The discovery of the cortisol:C-peptide ratio as a key biomarker, for instance, represents a separate inventive contribution from the discovery of the therapeutic composition itself. This metabolic mechanism operates in parallel with other control systems, including direct central nervous system control of the HPA axis by the relaxin-3/RXFP3 neuropeptide system, providing multiple pathways for stress axis dysregulation in a state of hormonal imbalance (McGowan et al., 2014).

### Advantages Over Prior Art:

A Unified Mechanism for Diabetic Complications: This invention is based on the discovery, supported by sophisticated artificial intelligence (AI) platforms, that C-peptide is a pleiotropic hormone orchestrating metabolic homeostasis through these distinct receptor interactions. The invention is further based on c-peptide serving as a diagnostic marker, with molecules like vitamin D studied in disease settings (Al-Qahtani F S, 2024; Chandler P D, 2015; Pittas A G, 2012). In the absence of C-peptide, unopposed insulin stimulation of the GPR146 receptor leads to chronic hypercortisolemia. This metabolic mechanism operates in parallel with other control systems, including direct central nervous system control of the HPA axis by the relaxin-3/RXFP3 neuropeptide system, providing multiple pathways for stress axis dysregulation in a state of hormonal imbalance (McGowan et al., 2014). The invention further provides for a companion diagnostic method to identify patients most likely to benefit from the therapy. This method is based on the discovery that the cortisol:C-peptide ratio serves as a direct quantitative measure of the pathological GPR146 signaling that the co-formulation is designed to correct, thereby enabling a precision medicine approach to treatment.

The Relaxin-3/RXFP3 system is recognized for its broad modulation of central nervous system functions, including appetite, stress responses, and behavior (McGowan et al., 2008; Smith et al., 2013; Tanaka M, et al., 2005; Watanabe Y, et al., 2010). This elevated cortisol induces a functional Vitamin D deficiency by disrupting its bioactivation. The resulting Vitamin D deficiency removes the physiological suppression of the renin gene (Li et al., 2004), leading to overactivation of the renin-angiotensin system (RAS). The overactive RAS, particularly its ACE/Angiotensin II/AT1R axis, is a primary driver of the pro-inflammatory and pro-fibrotic conditions underlying diabetic nephropathy and cardiovascular disease (Patel et al., 2016). The present invention corrects this entire cascade by restoring the C-peptide-mediated “off-switch” for GPR146 signaling. By normalizing SREBP2 activity, the invention not only corrects cholesterol biosynthesis but also indirectly restores fatty acid homeostasis through the SREBP2-LXR-SREBP-1c regulatory axis, thereby providing a comprehensive treatment for diabetic dyslipidemia. The invention's ability to correct this entire cascade is further supported by the molecular link between key metabolic sensors like SIRT1, which is modulated by C-peptide signaling, and the regulation of RAS components such as ACE2 (Clarke et al., 2014).

Superior Mass-Efficiency and Cost-Effectiveness: The coordinated action of the co-formulation results in a profound therapeutic synergy. In representative models for achieving equivalent glycemic control, the claimed composition is approximately 7-fold more mass-efficient than insulin monotherapy. This efficiency provides a powerful insulin-sparing effect, reducing the required therapeutic mass of the insulin component by over 10-fold and translating to a significant reduction in the overall cost of the active pharmaceutical ingredients.

A Superior Dual-Mechanism Therapy for Alzheimer's Disease: The present invention provides a superior method for treating or preventing Alzheimer's disease through a synergistic, dual-action mechanism that is not disclosed or suggested by the prior art. In addition to the disclosed neuronal pathway that prevents the production of amyloid-beta (Aβ) peptides, the invention leverages a second, distinct neuro-immune pathway that promotes the clearance of existing Aβ plaques. This is achieved by correcting the state of chronic hypercortisolemia associated with C-peptide deficiency. It is known that chronic stress and elevated corticosteroids are associated with the downregulation of the adhesion G protein-coupled receptor ADGRG1 (also known as GPR56) on microglia, the brain's resident immune cells (Piao et al., 2025). By normalizing cortisol levels, the co-formulation preserves the expression of ADGRG1 on microglia. This enables a protective microglial state driven by the activation of the transcription factor MYC, which promotes the phagocytosis and lysosomal degradation of Aβ plaques. This dual “pincer movement”—simultaneously inhibiting Aβ production and enhancing its clearance—represents a more robust and comprehensive therapeutic strategy for Alzheimer's disease than any single-mechanism approach.

Integrated Manufacturing Platform: Novel co-encapsulation technology maintains precise stoichiometric ratios during sustained release, solving degradation and delivery challenges that have limited previous combination attempts.

Precision Medicine Integration: The therapy allows for algorithmic patient selection based on multi-biomarker profiling, enabling personalized dosing and optimization.

The invention is intended to treat or prevent diseases and complications associated with C-peptide deficiency, including neuropathy, retinopathy, nephropathy, and cardiovascular disease, by restoring natural pancreatic hormone coordination. The invention is further applicable to the treatment or prevention of diabetes-associated fibrotic lung disorders such as pulmonary fibrosis (Ding Y., 2024), and metabolic wasting syndromes including sarcopenia and cachexia, which are driven by the inflammatory and catabolic states corrected by the claimed co-formulation (Pollakova D., 2023; James H., 2022; Shen Y., 2022)

Coordinated Multi-Receptor Activation: The co-formulation achieves binding to multiple receptors, including the insulin receptor (IR), GPR146, and the relaxin receptor (RXFP1), as well as other potential receptors on specific cell types such as immune cells, creating synergistic effects that are impossible with separate administration of insulin and C-peptide.

### The Role of Glucocorticoid Resistance (GCR) in Metabolic Disease

The progression of metabolic disorders, particularly type 1 and type 2 diabetes, is frequently compounded by a condition known as Glucocorticoid Resistance (GCR). Glucocorticoids (GCs), such as cortisol, are crucial stress hormones that modulate systemic energy metabolism, inflammation, and immunity. While acute GC signaling is essential for survival, chronic dysregulation or tissue insensitivity leads to pathological outcomes. GCR represents a state where the metabolic tissues (e.g., liver, muscle, and adipose tissue) lose appropriate sensitivity to the negative feedback of the hypothalamic-pituitary-adrenal (HPA) axis, leading to persistently elevated or hypercortisolemic states (Charmandari E, 2018; Chrousos G P, 2009).

This chronic hypercortisolemia is a powerful driver of key diabetic pathologies: it amplifies insulin resistance across target tissues, promotes hepatic gluconeogenesis, inhibits peripheral glucose uptake, and drives the central deposition of fat (Chrousos G P, 2009). Furthermore, this chronic neuroendocrine stress contributes significantly to pancreatic β-cell exhaustion and eventual failure.

Conventional diabetes therapies, including standard insulin replacement and glucose-lowering agents, primarily address the downstream consequences of metabolic failure. These treatments fail to restore the integrated neuroendocrine signaling network and, critically, do not possess the molecular mechanisms required to terminate the pro-metabolic signals that sustain GCR and persistent hypercortisolemia.

There is, therefore, a pressing and unmet need for a therapeutic agent that can restore the physiological coordination between the metabolic and neuroendocrine systems to achieve comprehensive, long-term diabetes management and prevent complication progression. The present invention addresses this critical need by utilizing the unique, multimodal signaling properties of C-peptide to modulate the HPA axis and terminate the pathological signals driving GCR.

While insulin replacement and sensitivity-enhancing agents exist, they primarily address the consequences of pancreatic and metabolic failure, failing to restore the native, integrated hormonal signaling network. Critically, these conventional approaches do not possess the molecular mechanisms required to modulate the dysregulated HPA axis or terminate the pro-metabolic signals responsible for GCR and persistent hypercortisolemia. There is, therefore, a pressing need for a therapeutic agent that can restore the physiological coordination of the metabolic and neuroendocrine systems to achieve comprehensive, long-term diabetes management and prevent complication progression.

### Companion Diagnostic for Patient Stratification

A key aspect of the present invention is a companion diagnostic method for patient stratification, designed to identify individuals with the specific hormonal dysregulation that the co-formulated composition targets. The diagnostic is based on quantifying the patient's cortisol:C-peptide ratio from a biological sample, such as blood, plasma, or serum.

Scientific Rationale: As disclosed herein, unopposed insulin stimulation of GPR146 drives hypercortisolemia via the SREBP2-cholesterol pathway. A state of C-peptide deficiency exacerbates this effect. Therefore, the cortisol:C-peptide ratio is not merely a correlative biomarker but a direct, functional readout of the pathological activity of the target pathway. A high ratio indicates a patient whose diabetic complications are significantly driven by this mechanism and who is therefore an ideal candidate for treatment with the co-formulation.

Clinical Utility: This diagnostic method allows for a precision medicine approach, moving beyond treating all diabetic patients uniformly. By selecting patients with a cortisol:C-peptide ratio above a predetermined threshold (e.g., a ratio in the upper quartile of the diabetic population), clinicians can predict a more robust therapeutic response, leading to improved clinical outcomes and more efficient healthcare resource allocation. This method is suitable for development as a laboratory-developed test (LDT) or a fully regulated in vitro diagnostic (IVD) kit.

### Supporting Therapeutic Mechanisms

### Validation of Predicted Binding Under Physiologically Relevant Conditions

These computationally-guided conditions. are consistent with recent findings demonstrating that AI-based protein structure prediction achieves “substantially improved accuracy over many previous specialized tools” and can predict interactions that traditional physics-based methods cannot detect (Abramson et al., 2024). Unlike prior art studies that failed to demonstrate C-peptide binding under standard experimental conditions, the present invention validates AI-predicted interactions under conditions that mirror the physiological co-formulation environment:
Binding assays performed in the presence of zinc ions and albumin, as required by the co-formulation
Simultaneous insulin and C-peptide availability, reflecting the natural pancreatic secretion state
Membrane preparations maintaining native lipid environments and receptor conformations
Dynamic flow conditions mimicking circulatory physiology rather than static incubation

The invention is based on key mechanistic discoveries of C-peptide's therapeutic effects when co-administered with insulin:

### Mechanism of Insulin Receptor Potentiation:

Molecular docking and simulation studies reveal a complex and nuanced interaction between C-peptide, insulin, and the Insulin Receptor (IR). The data demonstrates that while both C-peptide and insulin are potent ligands for the receptor, their binding is mutually inhibitory, meaning the presence of one negatively impacts the binding of the other. This dynamic provides the molecular basis for the “tag-team” mechanism of action where the hormones work sequentially rather than simultaneously on a single receptor. The simulations show that C-peptide binds to the insulin receptor with a strong binding free energy of −123.28 kcal/mol, indicating a highly stable and favorable interaction comparable to, and in this model even more favorable than, the binding energy of insulin itself (−79.65 kcal/mol). Both peptides, when bound individually, restrict the overall conformational flexibility of the IR and increase the number of stabilizing hydrogen bonds compared to the receptor's free form, confirming the formation of stable binary complexes. The key discovery lies in the analysis of the ternary complexes—where the receptor is pre-bound with one ligand before the other is introduced. Multiple lines of evidence confirm that this state is energetically unfavorable and unstable. First, the data shows that C-peptide and insulin bind to adjacent but overlapping regions on the IR, sharing a number of common interacting amino acid residues, which creates steric hindrance and competition. Second, the binding energy of the ternary complexes is significantly less favorable than that of the binary complexes, indicating that the pre-binding of either insulin or C-peptide hinders the subsequent binding of the other. Third, the number of stabilizing H-bonds decreases in the ternary complexes, which show more diverse and scattered conformations, indicating a loss of stability and a more disordered structure. In summary, the data consistently demonstrates that while C-peptide and insulin both bind effectively to the Insulin Receptor, they do so in a mutually exclusive manner, supporting a dynamic model of sequential action rather than simultaneous co-binding.

### A Unified “Triple-Hit” Mechanism for Complications Based on an Integrated Endocrine Network:

The therapeutic rationale for this invention is grounded in a novel, integrated view of endocrine regulation, where the relaxin, C-peptide, and cortisol systems converge to control metabolism, inflammation, and stress. The dysregulation of this network in diabetes creates a “triple-hit” pathological state that the co-formulation is uniquely designed to correct, comprising:
(1) hormonal dysregulation via the Cortisol-RAS cascade
(2) loss of direct anti-fibrotic protection via the RXFP1 pathway, and
(3) pathological biophysical stress via the GPR146-PIEZO1 mechanotransduction pathway.

Furthermore, these protective effects may be enhanced through the engagement of key metabolic regulators like SIRT1, which plays a critical role in preventing vascular pathologies (D'Onofrio et al., 2018).

### Beta-Cell Preservation Through Immune Modulation

C-peptide treatment slows the loss of islet cells in T1D by promoting the targeted immune exhaustion of autoreactive T-cells. This mechanism is supported by a transcriptomic meta-analysis of Type 1 Diabetes patient data (PMID: 33351781). Using Weighted Gene Co-expression Network Analysis (WGCNA), a specific module of 739 genes was identified whose expression shows a significant positive correlation (R=0.47, p<0.05) with C-peptide levels. These gene signatures are characteristic of exhausted CD8+ and CD4+ T-cells as well as regulatory T-cells (Tregs), indicating that higher C-peptide levels are associated with an immune-tolerant state. Further network analysis indicates that C-peptide signaling suppresses NF-κB and modulates the IL-2 pathway, subsequently upregulating immune checkpoint markers and key transcription factors like FOXP3. This helps preserve residual 3-cell function in patients, particularly those with new-onset T1D.

### Diabetic Nephropathy

The progression of kidney disease is explained by a “dual hit” mechanism. Hit 1 is primarily hyperglycemia itself. The initial drivers are:
Metabolic abnormalities (high blood sugar, AGEs).
Hemodynamic changes (intraglomerular hypertension from afferent arteriole dilation).

These initial hits then activate secondary pathways like RAS, TGF-β, and inflammatory cytokines. The presence of inflammatory markers can lead to higher levels of circulating cortisol, leading to glomerular hypertension and fibrosis via mediators like Transforming Growth Factor-beta (TGF-β). Hit 2 is the loss of protection, where the absence of C-peptide binding to RXFP1 prevents direct anti-fibrotic signaling, crippling the kidney's ability to counter the inflammatory assault. The binding of C-peptide to RXFP1 is particularly significant as it taps into a potent protective system. Molecular docking and simulation studies confirm this interaction, showing that C-peptide binds to the Relaxin Receptor with a favorable binding energy of −54.25 kcal/mol. While this is a lower affinity than the native ligand, Relaxin 3, principal component analysis demonstrates that C-peptide binding forms a stable complex that significantly reduces the receptor's conformational flexibility. The interaction is stabilized by 13 hydrogen bonds and 25 hydrophobic interactions and occurs at a neighboring region to the native ligand, sharing several key amino acid residues. Canonical signaling of RXFP1 involves G-protein-mediated activation of adenylyl cyclase, increasing intracellular cyclic AMP (cAMP), and engages the PI3K and MAPK/ERK cascades (Halls M L, 2006; Ahmad N, 2012). These pathways are responsible for powerful physiological effects including vasodilation, angiogenesis, and potent anti-fibrotic actions, which directly counteract the pathology of diabetic nephropathy. The co-formulation addresses both hits, suppressing T1D-induced nephropathy by down-regulating the TGF-β fibrosis cascade, a mechanism supported by evidence that C-peptide inhibits collagen synthesis by preventing SMAD3 binding to key gene promoters (Li Y, 2018). When pathologically upregulated in diabetic kidney disease, TGF-β drives fibrosis by promoting the synthesis of extracellular matrix proteins, inducing epithelial-to-mesenchymal transition (EMT), and activating myofibroblasts. The co-formulation's protective effect is achieved as C-peptide suppresses TGF-β through multiple mechanisms, including the reduction of oxidative stress, improvement of endothelial function, and direct inhibition of TGF-β gene transcription.

### Cardiovascular Disease

A similar “dual hit” mechanism underlies cardiovascular disease. The overactive RAS drives vasoconstriction and inflammation (Boels K, 2022). The lack of C-peptide signaling via IR and RXFP1 leads to impaired endothelial function, reduced nitric oxide (NO) bioavailability, and increased activity of the pro-inflammatory transcription factor NF-κB (Forst T., 1998; Kitamura T, 2003). The co-formulation provides cardioprotective effects through decreased endothelial permeability and suppression of NF-κB and other inflammatory mediators. Furthermore, these protective effects may be enhanced through the engagement of key metabolic regulators like SIRT1, which plays a critical role in preventing vascular pathologies (D'Onofrio N, 2018).

### Direct Modulation of the Glucocorticoid System

In addition to indirect control via the GPR146-SREBP2-cortisol substrate pathway, the integrated network reveals a direct interaction with the glucocorticoid system. Evidence shows that related peptides, such as relaxin-2, can act as direct agonists at the intracellular Glucocorticoid Receptor (GR), independent of any cell-surface receptor like RXFP1. This interaction was confirmed using a modified relaxin that was inactive at RXFP1 but still produced potent anti-inflammatory effects that were completely blocked by a GR antagonist (Dschietzig et al., 2009). This non-canonical, RXFP1-independent pathway allows for glucocorticoid-like anti-inflammatory effects separate from cell-surface signaling. It represents a parallel anti-inflammatory axis, providing signaling redundancy and more robust control over inflammation. The ability of C-peptide to interact with this broader relaxin/glucocorticoid network via RXFP1 suggests its anti-inflammatory and anti-fibrotic benefits may be more profound than previously understood, involving both canonical RXFP1 signaling and potential modulation of this direct GR pathway (Figueiredo et al., 2006). In addition to indirect control via the GPR146-SREBP2-cortisol substrate pathway, the integrated network reveals a direct interaction with the glucocorticoid system.

### The GPR146-CREB1-CBP-PIEZO1 Axis: A Dual-Function Regulator of Vascular Homeostasis

A central and profoundly impactful discovery of the present invention is the identification of a dual-function signaling axis that is pathologically dysregulated in C-peptide deficiency and precisely corrected by the co-formulated composition. This intricate axis, involving the G-protein coupled receptor GPR146, its distinct G-protein signaling cascades, the transcription factors CREB1 and CBP, and the mechanosensitive ion channel PIEZO1, acts as a critical molecular switch governing vascular health and disease progression.

In the pathological state prevalent during C-peptide deficiency, unopposed insulin stimulation of GPR146 on vascular smooth muscle and endothelial cells initiates a detrimental Gai-mediated signaling cascade (Chen Z et al., 2025). This Gai activation leads to a marked decrease in intracellular cyclic AMP (cAMP) levels. The reduction in cAMP impairs the activity of Protein Kinase A (PKA), which normally phosphorylates and activates CREB1. Consequently, CREB1 remains in an inactive state, unable to drive the expression of protective genes. Simultaneously, the sustained Gai signaling at GPR146 directly contributes to pathological changes in cell membrane biophysics, which in turn leads to the direct, aberrant activation of PIEZO1 (Chen Z et al., 2025; Ranade et al., 2015). This pathological activation of PIEZO1 results in a massive, uncontrolled influx of calcium ions (Ca²⁺) into vascular cells. This surge in intracellular Ca²⁺ is a direct trigger for sustained vasoconstriction, increased vascular stiffness, and the activation of pro-fibrotic gene expression programs through calcium-sensitive transcription factors (Bagher P et al., 2018; Du et al., 2021). These combined effects aggressively drive hypertension and vascular remodeling.

Furthermore, this pathological state is exacerbated by a devastating negative feedback loop. As established herein, unopposed insulin stimulation of GPR146 also drives hypercortisolemia. Critically, elevated cortisol levels are known to directly inhibit the expression of the CREB-binding protein (CBP), which contains a corticosteroid response element in its gene promoter (Nariai et al., 1997; Xu et al., 2012). CBP is an indispensable transcriptional coactivator, crucial for CREB1-mediated gene expression (Chrivia et al., 1993; Mayr and Montminy, 2001). Thus, high cortisol actively depletes the cellular machinery required to mount a protective transcriptional response, effectively creating a vicious cycle where the disease actively suppresses its own resolution.

The present invention acts by restoring the protective state of this critical axis. Co-administration of C-peptide in the formulation promotes the internalization of GPR146, terminating the pathological Gai signaling. More importantly, C-peptide simultaneously activates specific G-protein coupled receptors (likely through distinct Gas signaling pathways) that lead to a beneficial increase in intracellular cAMP. This rise in cAMP robustly activates Protein Kinase A, which then phosphorylates CREB1 (Mayr and Montminy, 2001). This phosphorylation is the essential activation switch for CREB1. Concurrently, by normalizing cortisol levels, the invention removes the inhibitory suppression on CBP expression, thereby restoring its cellular availability (Nariai et al., 1997). The now activated CREB1 efficiently recruits the restored CBP to form a potent CREB1-CBP complex (Chrivia et al., 1993). This complex then effectively binds to the promoter region of the PIEZO1 gene, initiating its protective transcription and ensuring its proper homeostatic regulation.

This C-peptide-driven upregulation of PIEZO1, within a context of normalized cellular signaling and cortisol levels, shifts PIEZO1 from its pathological overactivation to a state of homeostatic activation. In this controlled state, PIEZO1 contributes to beneficial mechanosensing, promoting physiological vasodilation, reducing vascular stiffness, and engaging anti-inflammatory pathways (Wu et al., 2020; Albarrán-Juárez et al., 2023). By simultaneously terminating the pathological GPR146/PIEZO1 activation, restoring CBP, and promoting homeostatic PIEZO1 expression, the present invention breaks the vicious cycle of vascular instability, directly reversing pathological remodeling, decreasing inflammation, and restoring vascular homeostasis.

### Dyslipidemia

The persistent dyslipidemia in diabetic patients is explained by the chronic, unopposed insulin or stimulation of GPR146. Without the C-peptide “off-switch,” sustained signaling through this receptor leads to chronic activation of the transcription factor SREBP2, resulting in the continuous overproduction of cholesterol and fatty acids by the liver. SREBP2 is the master transcriptional regulator of cholesterol homeostasis (Horton et al., 2002; Jeon and Osborne, 2012; Madison, 2016; Shimano and Sato, 2017). Its activation, driven by the insulin-GPR146-ERK axis, upregulates a suite of genes responsible for cholesterol and lipid synthesis, most notably HMG-CoA reductase (HMGCR), the LDL Receptor (LDLR), Fatty Acid Synthase (FASN), and Stearoyl-CoA Desaturase-1 (SCD1) (Yu et al., 2019).This pathway provides the molecular link between the hormonal signal (insulin) and the metabolic outcome (dyslipidemia). The excess cholesterol produced not only contributes to dyslipidemia but also serves as the obligate precursor for steroidogenesis, providing a direct substrate-driven mechanism for the hypercortisolemia observed in diabetes (Anagnostis et al., 2009). This GPR146-ERK-SREBP2 axis provides a single, unified molecular explanation for the dual comorbidities of dyslipidemia and hypercortisolemia.

This invention's mechanism is further strengthened by the discovery that SREBP2 acts as the essential upstream activator for SREBP-1c, the key lipogenic transcription factor. The chronic SREBP2 activity driven by unopposed insulin signaling generates an endogenous sterol ligand that activates the Liver X Receptor (LXR). Activated LXR is the primary transcriptional activator of the SREBP-1c gene (Shimano, 2021). Therefore, by correcting the primary defect—pathological SREBP2 activation—the co-formulation creates a powerful downstream effect, shutting down the LXR-mediated over-expression of SREBP-1c and halting the excessive fatty acid synthesis that drives hepatic steatosis and hypertriglyceridemia. This provides a comprehensive, single-point intervention for the full spectrum of diabetic dyslipidemia. The therapeutic effect is amplified as C-peptide signaling is known to activate AMPK and SIRT1, both of which are established negative regulators of SREBP processing and activity (Li et al., 2011), while the co-formulation's anti-inflammatory effects counter the pro-SREBP2 signaling driven by the mTOR pathway (Peterson et al., 2011).

### Diabetic Neuropathy

Peripheral neuropathy is attributed to the loss of C-peptide's direct protective effects on neural and vascular tissues. C-peptide is essential for maintaining the activity of the Na+/K+-ATPase pump in nerve cells (Wahren et al., 2012), a complex enzyme whose function is critical for proper nerve conduction and is subject to intricate redox-sensitive regulation. C-peptide also improves neural perfusion by enhancing microvascular blood flow (Bogdanova et al., 2016).

A Novel Protective Mechanism for the Na⁺,K⁺-ATPase Pump

The invention's protective effects on neural and vascular tissues are further explained by a novel protective mechanism for the Na⁺,K⁺-ATPase pump. In a state of C-peptide deficiency, the resulting hypercortisolemia drives the co-production of endogenous ouabain, a potent inhibitor of the pump. The administration of the claimed co-formulation restores nitric oxide (NO) production via eNOS activation. This NO subsequently causes the S-nitrosylation of specific cysteine residues on the Na⁺,K⁺-ATPase pump. This S-nitrosylation sterically hinders the binding of the inhibitory ouabain molecule, thereby preserving the pump's essential function in maintaining cellular ion gradients even in a state of hormonal dysregulation (Bogdanova et al., 2016).

### Prevention of a Pathological Metabolic Shift (The “Warburg Effect”)

Furthermore, the invention prevents a catastrophic metabolic shift in affected cells. In the C-peptide deficient state, chronic oxidative stress leads to the pathological activation of the non-receptor tyrosine kinase Src, which is part of the Na+,K+-ATPase signaling platform. Activated Src phosphorylates and inactivates the pyruvate dehydrogenase complex (PDC), the gatekeeper enzyme that directs pyruvate into the citric acid cycle for efficient energy production. With the PDC inactivated, cells are forced to shift toward aerobic glycolysis, also known as the “Warburg effect”. This metabolic reprogramming reduces ATP production efficiency and increases the generation of methylglyoxal, a precursor to advanced glycation end products that drive diabetic complications. By maintaining normal Na+,K+-ATPase function and preventing pathological Src activation, the co-formulation preserves normal oxidative metabolism and prevents this damaging metabolic shift.

### Alzheimer's Disease

C-peptide may play a crucial neuroprotective role. The proposed pathway begins with C-peptide binding to GPR146, causing internalization and through low levels of cortisol and unsuppressed vitamin D₃ conversion to calcitrol, upregulates SIRT1 via vitamin D response elements (VDREs). SIRT1 then exerts a dual benefit: it inhibits the amyloidogenic pathway by suppressing the BACE-1 enzyme that generates toxic amyloid-β (Aβ) peptides and concurrently promotes the non-amyloidogenic pathway by upregulating the ADAM10 enzyme, which precludes Aβ formation (Chen and Gong, 2019; Vassar, 2009). By preventing Aβ formation, C-peptide signaling may indirectly mitigate tau pathology. SIRT1 then exerts a dual benefit: it inhibits the amyloidogenic pathway by suppressing the BACE-1 enzyme that generates toxic amyloid-β (Aβ) peptides and concurrently promotes the non-amyloidogenic pathway by upregulating the ADAM10 enzyme, which precludes Aβ formation (Chen and Gong, 2019; Vassar, 2009).

### A Synergistic Dual-Mechanism Neuroprotective Pathway for Alzheimer's Disease

The present invention's therapeutic effect in Alzheimer's disease is not limited to the neuronal GPR146-SIRT1 pathway that prevents amyloid-beta (Aβ) production. The invention further provides a second, complementary neuro-immune mechanism that actively promotes the clearance of existing Aβ plaques, creating a superior, dual-action therapeutic approach. This second mechanism is initiated by the co-formulation's disclosed ability to correct the state of chronic hypercortisolemia that results from C-peptide deficiency. Chronic physiological stress and the associated elevation of corticosteroids are known to suppress the expression of Adhesion G Protein-Coupled Receptor G1 (ADGRG1, also known as GPR56). ADGRG1 is a receptor expressed on microglia, the resident innate immune cells of the central nervous system (Piao et al., 2025). The expression of ADGRG1 is critical for maintaining a protective microglial state. Signaling through ADGRG1 activates the transcription factor MYC, which in turn drives a transcriptomic program that enhances the microglial capacity for phagocytosis and lysosomal degradation of Aβ plaques.

In mouse models of Alzheimer's disease, a deficiency of ADGRG1 in microglia leads to a significant increase in Aβ plaque burden and accelerated cognitive decline. Conversely, higher levels of ADGRG1 in human microglia are associated with milder disease symptoms (Piao et al., 2025). By normalizing systemic cortisol levels, the administration of the claimed co-formulation prevents the stress-induced downregulation of ADGRG1. This preserves the protective, MYC-mediated Aβ clearance function of microglia. This novel and non-obvious link-connecting the correction of systemic hypercortisolemia to the preservation of a specific neuro-immune Aβ clearance pathway-forms the basis for the invention's superior, dual-mechanism therapeutic effect. The synergy between inhibiting Aβ production (via the GPR146-SIRT1 axis) and promoting Aβ clearance (via the Cortisol-ADGRG1-MYC axis) is summarized in Table 1.

TABLE 1
Comparative Analysis of Neuroprotective Mechanisms for Alzheimer's Disease

| | Mechanism 1 | Mechanism 2 | |
|---|---|---|---|
| Feature | (GPR146-SIRT1) | (ADGRG1-MYC) | Therapeutic Synergy |
| Primary | GPR146 | ADGRG1 | Targets two |
| Receptor | | (GPR56) | distinct, non- |
| | | | overlapping receptor |
| | | | systems, reducing the |
| | | | risk of pathway |
| | | | resistance. |
| Primary | Neurons | Microglia | Engages both |
| Cell Type | | (resident brain | the central nervous |
| | | immune cells) | system and its innate |
| | | | immune system for a |
| | | | comprehensive |
| | | | therapeutic effect. |
| Key | SIRT1 (a | MYC (a | Activates two |
| Molecular Mediator | deacetylase) | transcription factor) | different classes of |
| | | | powerful intracellular |
| | | | regulators, broadening |
| | | | the biological response. |
| Effect on | Inhibits | Promotes | Creates a |
| Amyloid-β (Aβ) | Production: | Clearance: Enhances | “pincer movement” that |
| | Suppresses BACE-1 | microglial | simultaneously reduces |
| | and promotes non- | phagocytosis and | the influx of new Aβ |
| | amyloidogenic APP | lysosomal degradation | and removes existing |
| | processing. | of Aβ plaques. | Aβ aggregates. |
| Therapeutic | Reduces the | Clears existing | Provides a |
| Outcome | formation of new Aβ | pathological Aβ | powerful, dual-action, |
| | plaques. | deposits. | disease-modifying |
| | | | therapy expected to be |
| | | | superior to single- |
| | | | mechanism approaches. |

### Beta-Cell Preservation Through Immune Modulation

C-peptide treatment slows the loss of islet cells in T1D by promoting the targeted immune exhaustion of autoreactive T-cells (Al-Trad et al., 2015). Transcriptomic analysis indicates that C-peptide signaling suppresses NF-κB and subsequently upregulates immune checkpoint markers like PD-1 and TIGIT. This could help preserve residual β-cell function in patients with new-onset T1D.

### Mechanism of Action in Metabolic Syndrome and MAFLD/NASH

The therapeutic utility of the invention extends beyond diabetic complications to other related metabolic disorders, most notably Metabolic Syndrome (MetS) and Metabolic-Associated Fatty Liver Disease (MAFLD). The core pathology of these conditions is directly linked to the GPR146-SREBP2 signaling axis that this invention corrects.

Metabolic Syndrome is a complex disorder driven by interconnected pathologies, including insulin resistance, dyslipidemia, central obesity, and hypertension. The proposed invention targets the root cause of these issues by addressing two of its most powerful drivers at a single molecular checkpoint.

The core mechanism is based on the hypothesis that C-peptide restores physiological control over the GPR146 receptor, acting as a crucial ‘off-switch.’ This action directly inhibits the chronic activation of SREBP2, a master regulator responsible for synthesizing cholesterol and fatty acids. The primary therapeutic outcome is the normalization of lipid production, directly counteracting the dyslipidemia that is central to Metabolic Syndrome.

Furthermore, by reducing the overproduction of cholesterol—the essential precursor for cortisol—this intervention mitigates the effects of hypercortisolemia. As established by Pivonello et al. (2016), even mild elevations in cortisol are a powerful driver of insulin resistance and central obesity. By interrupting this vicious cycle, the co-formulation is positioned to correct multiple components of Metabolic Syndrome, moving beyond symptom management to address its underlying metabolic dysfunction.

MAFLD and its inflammatory progression, MASH, are characterized by the excessive accumulation of fat in the liver (hepatic steatosis). The metabolic dysfunction-associated fatty liver disease (MAFLD) and its inflammatory progression, metabolic dysfunction-associated steatohepatitis (MASH), are defined by the pathological accumulation of fat in the liver, known as hepatic steatosis. A recently discovered mechanism significantly clarifies the cause of this fat accumulation.

In states of insulin resistance, the gut hormone cholesin is over-secreted. This cholesin continuously stimulates its receptor, GPR146, in the liver. According to foundational research by Hu et al. (2024), this signaling axis directly drives the “continuous overproduction of cholesterol and fatty acids” by activating the master metabolic regulator, SREBP2.

This finding establishes that dysregulation of the cholesin-GPR146 pathway is a direct cause of the excessive fat synthesis underlying MAFLD. Consequently, a therapeutic composition designed to restore physiological control over this specific pathway would be uniquely positioned to treat or prevent MAFLD and halt its progression to MASH.

C-peptide has been used as a surrogate marker for monitoring the course of type 1 and type 2 diabetes and determining the effects of interventions designed to preserve and improve residual β-cell function. Current versions of insulin sold on the market have had the C-peptide genetically engineered to be as short as possible, as it was originally thought to be only important for ensuring the proper folding to align the A and B peptide chains to have the correct disulfide bonds form. Cebix tried to develop a genetically engineered C-peptide having improved bioavailability designed to combat loss of nerve function tied to Type 1 diabetes. In their Phase IIb trial, Ersatta (pegylated C-peptide) failed to show improved nerve function relative to the placebo. Described herein is a composition comprising insulin and C-peptide at a ratio of (0.8-1.2 parts of C-peptide to 1 part of insulin).

According to an aspect of the invention, there is provided a fast-acting composition comprising a co-formulation of C-peptide and insulin, wherein the C-peptide is added at 0.8-1.2 parts per 1 part insulin. From a formulation standpoint, combining insulin with C-peptide provides a carrier molecule effect by preventing aggregates from forming upon resuspension of a lyophilized pellet, as discussed herein. In some embodiments of the invention, the human C-peptide peptide or protein comprises or consists of or consists essentially of the amino acid sequence as set forth in SEQ ID No: 3: EAEDLQVGQVELGGGPGAGSLQPLALEGSLQ.

In some embodiments of the invention, the human insulin protein comprises or consists of or consists essentially of two chains, A and B, linked by disulfide bonds, having the amino acid sequences as set forth in SEQ ID No: 1 and SEQ ID No: 2, respectively: A-chain: GIVEQCCTSICSLYQLENYCN (SEQ ID No: 1) and B-chain:

(SEQ ID No: 2)
FVNQHLCGSHLVEALYLVCGERGFFYTPKT.

The composition requires a suitable buffer; for example, a sodium phosphate buffer. C-peptide has been shown to interact with insulin and form precipitates at pH at or below 6.0. For example, a pH 7.4 PBS solution is suitable for dissolving insulin and C-peptide, although other suitable buffers will be readily apparent to one of skill in the art and can be tested using routine. The composition may further comprise at least one suitable cryoprotectant, for example, trehalose and/or sorbitol, and a stabilizing agent, such as a zinc compound (e.g., zinc acetate, zinc chloride). These zinc salts help in forming insulin hexamers, which support stability and controlled release. For a lyophilized formulation, the ideal bulking agent should provide stability, maintain the integrity of the peptides, and ensure a good cake structure; examples include mannitol, glycerol, glycine, sucrose, trehalose, and sorbitol.

In some embodiments of the invention, there is provided a fast-acting composition comprising: Insulin: 90-110 or about 100 U/mL; C-Peptide: at a ratio of 0.8-1.2 parts per 1 part insulin, for example, about 1 mg/mL; Zinc Chloride: 0.05-0.3 or about 0.1 mg/mL; Trehalose: 3-7% or about 5% (w/v); Sorbitol: 2-4% or about 3% (w/v); Sodium Phosphate Buffer: 10 mM, pH 7.4; and Glycine: 1-3% or about 2% (w/v). In other embodiments to be lyophilized, the composition further comprises one or more of: a suitable amino acid, such as arginine or histidine; a sugar or polyol such as sucrose or mannitol; a surfactant such as polysorbate 80; and an antioxidant such as ascorbic acid. For example, in some embodiments, glycine is added at 0.5 to 2% (w/v), polysorbate 20 between 0.01% and 0.1% (w/v), and methionine is added as an antioxidant (0.1 to 1% (w/v)).

In another embodiment, there is provided a fast-acting composition comprising: Insulin: 90-110 or about 100 U/mL; C-Peptide: at a ratio of 0.8-1.2 parts per 1 part insulin, for example, about 1 mg/mL; Zinc Chloride: 0.05-0.3 or about 0.1 mg/mL; Trehalose: 3-7% or about 5% (w/v); Sorbitol: 2-4% or about 3% (w/v); Sucrose: 3-7% or about 5% (w/v); Sodium Phosphate Buffer: 10 mM, pH 7.4; Glycine: 0.05-3% or about 2% (w/v); Mannitol: 2-4% or about 3% (w/v); Arginine: about 1% (w/v); Histidine: about 1% (w/v); Polysorbate 80: about 0.05% (w/v); and Ascorbic Acid: about 0.1% (w/v).

In other embodiments of the invention, there is provided a slow-release formulation comprising 0.8-1.2 parts C-peptide to 1 part insulin. For example: Insulin: 90-100 or about 100 U/mL; C-Peptide: at a ratio of 0.8-1.2 parts per 1 part insulin, for example, about 1 mg/mL; PLGA (Poly(lactic-co-glycolic acid)): 8-12 or about 10 mg/mL; Zinc Chloride: about 0.1 mg/mL; Sucrose: 3-7% or about 5% (w/v); Mannitol: 2-4% or about 3% (w/v); Sodium Phosphate Buffer: 10 mM, pH 7.4; and Glycine: 0.05-3% or about 2% (w/v). In some embodiments, Insulin and C-peptide are dissolved in a buffer, and then added to an organic solvent layer containing PLGA, which is then added to a bulk water agent. The PLGA encapsulates the insulin and C-peptide. This mixture can be for intermuscular injection or the PLGA can be decorated with appropriate lipids and folate to survive stomach acids and bind to folate receptors for internalization.

Diabetics generally take insulin twice per day with a fast acting and longer-term release, usually in the morning before breakfast and in the evening before supper. Fast acting insulin reaches peak effectiveness in about 1.5 to 2.5 hours and generally lasts for about 4 to 6 hours, while slow acting insulin reaches peak effectiveness after about 18-24 hours. Given the differences in half-life, fast acting C-peptide will persist in the body much longer than insulin. By combining insulin and C-peptide into a slow acting formulation, the release time will depend upon the particle size, which can be determined to last for a particular time frame (18-24 hours). In some embodiments, the fast acting and slow acting forms of the co-formulated composition are mixed at ratios of about 50%/50% or about 30%/70% (v/v) based upon individual requirements.

A person in need of such treatment is a Type 1 diabetic or a Type 2, insulin-dependent diabetic. Insulin is generally taken 15 to 20 minutes prior to eating and dosed for weight and the anticipated volume of carbohydrates. The total daily insulin requirement is typically between 0.5 to 1.0 unit per kilogram of body weight for Type 1 diabetics. For Type 2 insulin-dependent diabetics, initial doses might start at 0.1 to 0.2 units/kg/day and are adjusted based on blood glucose levels.

Lyophilized formulations will preferably be produced, but sterile liquid formats with an appropriate buffered system are also possible. The lyophilized product can be resuspended in water, for example, using Bactericidal Water for Injection (BWFI), which contains 0.9% Benzaldehyde as a bacteriostatic agent. Benzaldehyde could also be added into the formulation buffer prior to lyophilization for longer-term stability. The lyophilized product stored at −20° C. is stable over a 2-to-5-year period, whereas current liquid insulin formulations have a maximum shelf life of 12-16 months.

In other embodiments of the invention, there is provided use of the insulin-C-peptide co-formulated composition to treat or prevent or delay progression or delay onset of one or more symptoms of diseases or syndromes associated with low levels of circulating C-peptide, said diseases or syndromes being selected from the group consisting of cancer, Alzheimer's disease, cardiovascular disease, neuropathy, retinopathy and nephropathy.

The invention is suitable for large-scale pharmaceutical manufacturing using standard lyophilization and microsphere encapsulation techniques. The formulation is stable under refrigerated conditions for at least 12 months and can be distributed in both liquid and dried formats, making it viable for global deployment.

### Mathematical Basis for Total Efficiency Factor of C-Peptide

A central and commercially significant advantage of the present invention is the dramatic improvement in mass efficiency compared to insulin monotherapy.

Total Efficacy Factor (TEF) for C-peptide:

$$TEF\_c = ( MW\_insulin / MW\_ C - peptide ) \times ( \vert BA\_C - peptide \vert / \vert BA\_insulin \vert ) \times ( t 1 / 2 \_ C - peptide / t 1 / 2 \_insulin )$$

$$TEF\_c = ( 5.81 kDa / 3.02 kDa ) \times ( 128.23 kcal / mol / 79.65 kcal / mol ) \times ( 30 min / 5 min )$$

$$TEF\_c = 1.9 24 \times 1.610 \times 6.0 = 18.58$$

Where:

$$MW = Molecular Weight$$

$$BA = Binding Affinity$$

$$t 1 / 2 = Half - life$$

### Dosing Formulas for Co-Formulation

1. Total Therapeutic Requirement (in Units):

$$U\_total = U\_meal + U\_correction$$

$$U\_total = ( S / 15 ) + ( ( BG\_current - BG\_target ) / I S F )$$

Where:

$$S = sugar to clear ( grams )$$

$$BG\_current = current blood glucose ( mg / dL )$$

$$BG\_target = target blood glucose ( mg / dL , typically 100 )$$

$$I S F = Insulin Sensitivity Factor ( mg / dL per Unit )$$

2. Component Contributions (based on equimolar dosing):

The total effect is split 9.4% insulin/90.6% C-peptide:

$$U\_insulin = U\_total \times 0.094$$

$$U\_ C - peptide = U\_total \times 0.906$$

3. Mass Calculations:

$$m\_insulin ( μg ) = U\_insulin \times 34.7$$

$$m\_ C - peptide ( μg ) = m\_insulin \times 0.52$$

$$m\_total ( μg ) = m\_insulin + m\_ C - peptide$$

Simplified:

$$m\_total ( μg ) = S \times 0.3 \; 34 ( for meal coverage only )$$

### Example Calculation

$$Scenario : Clear 30 g sugar , BG\_current = 225 mg / dL , ISF = 50$$

### Step 1: Calculate Total Units Required

$$U\_total = ( 30 / 15 ) + ( ( 225 - 100 ) / 50 )$$

$$U\_total = 2. + 2.5 = 4.5 Units$$

### Step 2: Calculate Component Masses

Co-formulation:

$$m\_insulin = ( 4.5 \times 0.094 ) \times 34.7 = 0.423 \times 34.7 = 14.68 μg$$

$$m\_ C - peptide = 14.68 \times 0.52 = 7.63 μg$$

$$m\_total = 14.68 + 7.63 = 22.31 μg$$

Standard insulin-only therapy:

$$m\_insulin - only = 4.5 Units \times 34.7 μg / Unit = 156.15 μg$$

### Step 3: Calculate Efficiency Improvement

$$Efficiency Ratio = m\_insulin - only / m\_total$$

$$Efficiency Ratio = 156.15 / 22.31 = 7. - fold$$

### Mass Efficiency Analysis

Insulin dose reduction:

$$Insulin Reduction = m\_insulin - only / m\_total ( in co - formulation )$$

$$Insulin Reduction = 156 \text{.15} / 14.68 = 10.64 - fold$$

Overall therapeutic efficiency:

$$Total Mass Efficiency = 156.15 μ g / 22.31 μ g = 7. - fold improvement$$

### Derivation of the 0.094/0.906 Split

This comes from solving the equimolar constraint with the efficacy factor:

$$At equimolar ratio : n\_insulin = n\_ C - peptide$$

$$Therefore : m\_ C - peptide = m\_insulin \times ( MW\_ C - peptide / MW\_insulin ) = m\_ C - peptide = m\_insulin \times ( 3 \text{.02} / 5.81 ) = m\_insulin \times 0. 52$$

Total effect equation:

$$S / 15 \times 34 \text{.7} = m\_insulin + ( m\_ C - peptide \times TEF\_c )$$

Substituting:

$$S / 15 \times 3 4.7 = m\_insulin + ( m\_insulin \times 0. 52 \times 18.58 )$$

$$S / 15 \times 34 \text{.7} = m\_insulin \times ( 1 + 9.66 )$$

$$S / 15 \times 34 \text{.7} = m\_insulin \times 10.66$$

Therefore:

$$m\_insulin = ( S / 15 \times 34.7 ) / 10.66$$

The insulin contribution to total effect:

$$Insulin fraction = 1 / ( 1 + 9.66 ) = 1 / 10.66 = 0.094 = 9.4 \%$$

$$C - peptide fraction = 9.66 / 10.66 = 0.096 = 90.6 \%$$

This mathematical framework demonstrates why the co-formulation achieves 7-fold mass efficiency while reducing the insulin dose by over 10-fold.

This 7-fold mass efficiency directly translates to multiple commercial and clinical advantages: (1) reduced cost of active pharmaceutical ingredients per dose, (2) smaller injection volumes improving patient comfort and compliance, (3) reduced cold-chain storage requirements due to lower mass per dose, (4) extended supply from the same quantity of raw materials, and (5) lower systemic drug exposure reducing potential immunogenicity. The insulin-sparing effect is even more pronounced, with the insulin component reduced by 10.6-fold (156.15/14.68), which is particularly significant given the manufacturing costs and immunogenic potential of recombinant insulin.

PLGA Particle Size and Release Duration: To support various therapeutic regimens, the particle size of PLGA microspheres is optimized to control the duration of protein release. Empirical data indicate that PLGA particles sized between 200-400 nm provide a sustained release of 12-18 hours, particles between 400-1000 nm support 18-24 hour release, and particles in the range of 5-20 μm enable a 1-week depot release. These ranges are influenced by polymer composition, molecular weight, porosity, and encapsulation method.

Transcriptomic meta-analysis and WGCNA clustering reveal that C-peptide treatment is associated with upregulation of immune exhaustion markers in CD8+ T cells and regulatory T cells. These findings support a mechanistic role for C-peptide in promoting immune tolerance via NF-κB suppression and downstream modulation of IL-2 and FOXP3 pathways.

This invention provides a novel pharmaceutical composition comprising a co-formulation of insulin and C-peptide designed to restore the natural physiological coordination of these hormones through simultaneous delivery. The composition comprises human insulin and human C-peptide, with C-peptide present at a ratio of 0.5 to 2.0 parts for every 1 part of insulin by molar ratio to mimic natural secretion. By delivering both hormones together, the formulation addresses critical gaps in current diabetes therapy.

The therapeutic efficacy is based on a mechanism of action involving coordinated multi-receptor activation. The simultaneous presence of both molecules allows C-peptide to not only enhance insulin signaling at the insulin receptor (insulinomimetic effects) but also to independently activate other key receptors like GPR146 and the relaxin receptor. This triggers a cascade of anti-inflammatory and vascular protective pathways that provide comprehensive benefits beyond simple glucose control.

This synergistic action provides targeted protection against diabetic complications. In diabetic nephropathy, C-peptide suppresses the TGF-β pathway, a key driver of kidney fibrosis (Li et al., 2018). For cardiovascular protection, it inhibits NF-κB, reduces inflammation, and improves vascular health. In diabetic retinopathy, it improves microvascular blood flow and maintains endothelial cell integrity. For peripheral neuropathy, it improves neural blood flow and corrects cellular pump deficiencies (Wahren et al., 2012). Furthermore, in Type 1 Diabetes, the co-formulation helps preserve remaining beta-cells by promoting targeted immune exhaustion. By restoring this crucial partnership, the invention offers a more holistic approach to managing diabetes and mitigating its long-term complications.

Furthermore, the therapeutic utility of the invention extends to other systemic complications of Type 1 Diabetes. The potent anti-fibrotic effects, mediated by the suppression of the TGF-β pathway and activation of RXFP1 signaling, provide a direct mechanism for treating or preventing the progression of pulmonary fibrosis, a recognized complication driven by chronic hyperglycemia and inflammation (Ding Y, 2024). Additionally, the co-formulation's ability to correct hypercortisolemia, restore normal oxidative metabolism, and prevent pathological metabolic shifts provides a powerful intervention for muscle wasting syndromes. By blunting the catabolic state and restoring the anabolic signals necessary for muscle maintenance, the invention directly addresses the underlying drivers of sarcopenia and the severe cachexia observed in states of absolute insulin deficiency (Pollakova D, 2023; James H, 2022; Shen Y, 2022).

In certain embodiments, the pharmaceutical composition further comprises recombinant human serum albumin (rHSA) at a concentration of at least 10 mg/L, preferably 1 mg/mL, in either liquid or lyophilized formulations. The inclusion of rHSA serves multiple purposes: it acts as a carrier in the bloodstream, enhances protein stability, and prevents aggregation of the active pharmaceutical ingredients. This excipient is particularly beneficial in maintaining the structural integrity and bioactivity of the co-formulated insulin and C-peptide during storage and administration.

### Definitions

“human insulin”: as used herein refers to the mature form of the hormone, comprising the A-chain as set forth in SEQ ID NO: 1 and the B-chain as set forth in SEQ ID NO: 2, connected by disulfide bonds, or functional analogs thereof. Functional analogs include, but are not limited to, variants having at least 90% sequence identity to SEQ ID NO: 1 and SEQ ID NO: 2 that retain at least 70% of the binding affinity to the insulin receptor as measured by a competitive radioligand binding assay, or variants modified to alter their pharmacokinetic profile without materially compromising their primary metabolic function. “such analogs”: may include, for example, conservative amino acid substitutions in non-critical binding regions, such as the substitution of a leucine for an isoleucine or an aspartic acid for a glutamic acid, which do not materially alter the protein's three-dimensional structure or function

“human C-peptide”: refers to the connecting peptide as set forth in SEQ ID NO: 3, or functionally equivalent variants thereof. Functionally equivalent variants include, but are not limited to, variants having at least 90% sequence identity to SEQ ID NO: 3 that retain the ability to induce GPR146 receptor internalization upon binding, or variants that demonstrate binding affinity to the RXFP1 receptor sufficient to activate anti-fibrotic signaling pathways. Such modifications may enhance stability, solubility, or bioavailability without compromising the critical receptor interactions disclosed herein.

“Functionally equivalent variants” may include modifications that enhance stability or solubility without compromising receptor binding, such as the substitution of non-critical amino acids or the addition of stabilizing motifs.

“physiologically relevant conditions”: as used herein refers to experimental conditions that mimic the in vivo environment of the co-formulated composition, including but not limited to: pH 7.2-7.6, presence of zinc ions (0.01-0.1 mM), albumin (0.8-1.2 mg/mL), physiological temperature (37° C.), and simultaneous availability of both insulin and C-peptide in the claimed molar ratios.

“computationally-guided conditions”: refers to the use of machine learning algorithms, molecular dynamics simulations, and computational modeling to identify protein-ligand interactions that are not detectable by conventional experimental approaches. Further details of the discovery methodology are provided in Appendix I. Recent scientific validation demonstrates that AI-based approaches achieve at least 50% improvement in accuracy over traditional physics-based methods for biomolecular structure prediction (Abramson et al., 2024), particularly under non-physiological experimental conditions where conventional approaches fail.

“simultaneous delivery” or “simultaneous administration”: as used herein refers to the administration of insulin and C-peptide in a manner that results in their co-presence at target cell receptors for a duration sufficient to enable the synergistic ‘tag-team’ effect on the Insulin Receptor and the coordinated on/off signaling at the GPR146 receptor. This is typically achieved when the peak plasma concentrations of both exogenously administered peptides occur within 30 minutes of each other, and preferably within 15 minutes.

“More broadly”, the term refers to the administration of insulin and C-peptide in a manner sufficient to allow C-peptide to modulate GPR146 signaling before the pro-metabolic effects of unopposed insulin become pathologically sustained.

ADGRG1: Adhesion G Protein-Coupled Receptor G1 (also known as GPR56), a receptor on microglia involved in the clearance of amyloid-beta plaques.

AMPK: AMP-activated protein kinase, a key metabolic sensor enzyme involved in cellular energy homeostasis.

BACE-1: Beta-secretase 1, an enzyme that generates toxic amyloid-β (Aβ) peptides, which is a key target in the Alzheimer's disease pathway.

Biased Agonism: A process where a ligand directs a receptor to favor one signaling pathway over another, such as preferentially inducing a 0-arrestin-mediated pathway.

C-peptide: Connecting peptide, a hormone that is co-secreted with insulin from the pancreas.

ECM: Extracellular Matrix, the network of proteins and other molecules that provide structural and biochemical support to surrounding cells.

eNOS: Endothelial Nitric Oxide Synthase, an enzyme responsible for producing nitric oxide (NO) in blood vessels, which is critical for vascular health.

GR: Glucocorticoid Receptor, an intracellular receptor that mediates the effects of glucocorticoids like cortisol and can be targeted to produce anti-inflammatory effects.

GPR146: G-protein coupled receptor 146, a key receptor for both C-peptide and insulin that regulates cholesterol and cortisol production.

HPA Axis: Hypothalamic-Pituitary-Adrenal axis, the body's central stress response system that controls the production of cortisol.

IR: Insulin Receptor, the primary cell-surface receptor that binds insulin to initiate glucose uptake and other metabolic actions.

MAFLD: Metabolic-Associated Fatty Liver Disease, a metabolic disorder involving fat accumulation in the liver, which can be treated by the invention.

MASH: Metabolic-Associated SteatohepatitisMetabolic-Associated SteoHepatitis

NF-κB: Nuclear Factor kappa-light-chain-enhancer of activated B cells, a protein complex that controls the transcription of DNA and is involved in inflammatory responses.

PD-1: Programmed cell death protein 1, an immune checkpoint marker whose upregulation is associated with the exhaustion of autoreactive T-cells.

PLGA: Poly(lactic-co-glycolic acid), a biodegradable polymer used to create sustained-release microspheres for drug delivery.

RAS: Renin-Angiotensin System, a hormonal system that regulates blood pressure and is a primary driver of pro-inflammatory conditions in diabetes when overactivated.

ROS: Reactive Oxygen Species, chemically reactive molecules containing oxygen that can cause cellular damage (oxidative stress) when produced in excess.

RXFP1: Relaxin Family Peptide Receptor 1, also known as the relaxin receptor, which C-peptide binds to activate anti-fibrotic pathways.

RXFP3: Relaxin Family Peptide Receptor 3, a receptor in the central nervous system involved in the neuroendocrine modulation of the stress axis.

SEC-HPLC: Size Exclusion Chromatography—High Performance Liquid Chromatography, an analytical technique used to assess formulation stability.

SIRT1: Sirtuin 1, a deacetylase enzyme that plays a crucial role in neuroprotection and the regulation of the RAS.

SMC: Smooth Muscle Cell, a type of cell found in blood vessel walls whose proliferation contributes to atherosclerotic plaques.

SREBP2: Sterol Regulatory Element-Binding Protein 2, a transcription factor that is the master regulator of cholesterol and fatty acid synthesis.

TGF-β: Transforming Growth Factor-beta, a signaling protein that is a key driver of fibrosis (scarring) in diabetic nephropathy.

TIGIT:T: cell immunoreceptor with Ig and ITIM domains, an immune checkpoint marker associated with T-cell exhaustion.

WGCNA: Weighted Gene Co-expression Network Analysis, a bioinformatics method used to identify gene modules associated with C-peptide levels.

### Formulation Stability and Key Optimizations

The long-term stability of the co-formulation is paramount and is achieved through a multi-component excipient system.

pH Stability: The formulation is maintained at a pH from 7.2 to 7.6 using a pharmaceutically acceptable agent, such as histidine, which is critical in formulations containing zinc as it avoids the precipitation that occurs with phosphate-based agents.

To protect against metal-catalyzed oxidation, a low-level chelating agent such as ethylenediaminetetraacetic acid sodium salt, or EDTA disodium salt, at a concentration from 0.01% to 0.05% is included.

Chemical Stability (Anti-Oxidation): To prevent oxidative damage, the formulation includes a sacrificial antioxidant, preferably L-methionine, at a concentration from 1 mg/mL to 3 mg/mL, which protects native methionine residues on both proteins.

Physical Stability (Lyophilized Cake Integrity): For lyophilized (freeze-dried) formulations, the physical structure is optimized with a weight ratio of an amorphous cryoprotectant (e.g., trehalose) to a crystalline bulking agent (e.g., mannitol) in a range of 3:1 to 10:1, with a preferred ratio of approximately 5:1, to provide a robust cake with maximal protein protection.

While specific excipients are described for the preferred embodiments, it is understood that other pharmaceutically acceptable agents within each functional class may be used. For example, the histidine buffer may be replaced with other physiological buffers such as Tris or citrate, provided they do not cause precipitation with other components. The sacrificial antioxidant L-methionine may be substituted with other antioxidants such as ascorbic acid, N-acetylcysteine, or monothioglycerol. Similarly, the chelating agent EDTA may be replaced with other agents like citric acid or deferoxamine to protect against metal-catalyzed oxidation.

Advanced Manufacturing for Sustained-Release Formulations: The manufacture of a sustained-release formulation comprising poly(lactic-co-glycolic acid) (PLGA) microspheres incorporates several novel steps to ensure stability and performance.

Protein Protection During Encapsulation: To protect the proteins from denaturation during emulsification, a pre-stabilization step is employed where the insulin and C-peptide are first complexed with zinc in an aqueous phase.

Stoichiometric Co-Encapsulation: To guarantee a consistent insulin-to-C-peptide ratio in each microsphere, a microfluidics-based manufacturing process is preferred over traditional batch homogenization.

Burst Release Control: A dangerous initial “burst release” of insulin is minimized by (a) washing the formed microspheres to remove loosely adsorbed surface protein, and (b) using a blend of PLGA polymers to slow initial water ingress and drug diffusion.

### Coordinated Therapeutic Mechanisms

Diabetic Retinopathy: C-peptide plays a vital role in protecting the retinal microvasculature. It enhances microvascular blood flow and reduces vascular permeability, which helps maintain the structural and functional integrity of the retina (Forst et al., 1998). Additionally, it supports endothelial cell function, decreases the production of reactive oxygen species (ROS), and limits extracellular matrix (ECM) deposition. These combined effects help slow the progression of retinal damage and vision loss in diabetic patients.

Microvascular Disease: Beyond specific organs, C-peptide's endothelial-protective effects extend to systemic microvascular networks. It has been shown to improve perfusion in both cerebral and peripheral microcirculation. These benefits may help prevent or reduce damage to small vessels, such as those in the brain and extremities, which are often compromised in diabetes and contribute to complications like cognitive decline and tissue ischemia.

Macrovascular Disease: In macrovascular complications, C-peptide demonstrates strong anti-inflammatory and antifibrotic properties. It protects mitochondrial function and inhibits the migration and proliferation of vascular smooth muscle cells (SMCs), which are key contributors to the development of atherosclerotic plaques. Furthermore, C-peptide supports endothelial function, helping to maintain vascular tone, reduce thrombosis risk, and improve overall vascular health.

Diabetic Kidney Disease: C-peptide exerts multiple protective effects on the kidneys. It regulates endothelial function and provides anti-inflammatory and antifibrotic benefits. Specifically, it helps prevent and attenuate glomerular hyperfiltration, reduces the diameter of afferent arterioles, inhibits tubular sodium reabsorption, and lowers levels of microalbuminuria. These actions are complemented by its ability to reduce renal inflammation, collectively contributing to the preservation of kidney function and slowing the progression of diabetic nephropathy (Wahren et al., 2012).

Diabetic Peripheral Neuropathy: In the peripheral nervous system, C-peptide perfusion and corrects deficiencies in Na+/K+-ATPase activity (Wahren et al., 2012). These effects are essential for maintaining proper nerve conduction and preventing axonal degeneration. By restoring these physiological processes, C-peptide may alleviate symptoms and improve nerve function in individuals with diabetic neuropathy.

Alzheimer's Disease: C-peptide may offer neurovascular protection in Alzheimer's disease by preserving the integrity of the blood-brain barrier and reducing neuroinflammation. It may also help mitigate vascular damage associated with amyloid accumulation. These effects are particularly significant given the growing recognition of vascular contributions to Alzheimer's pathology, suggesting a potential role for C-peptide in slowing disease progression.

Cardiovascular Disease: In cardiovascular disease, C-peptide targets several key pathological mechanisms. It helps restore endothelial function, reduces chronic inflammation, and limits oxidative stress. Additionally, it supports mitochondrial health and may prevent neointima formation, which is involved in vascular remodeling and plaque development. These actions collectively contribute to improved cardiovascular outcomes and reduced risk of atherosclerosis.

The invention will now be explained and/or elucidated by way of examples; however, the invention is not necessarily limited to or by the examples.

### EXAMPLES

### Example 1—Co-Formulation of Insulin and C-Peptide

Protocol: Human insulin is dissolved in an acidic aqueous solution, which is then neutralized. Human C-peptide is dissolved in a separate aqueous solution. The two solutions are combined in a desired ratio (e.g., 0.5:1 to 2.0:1 by molar ratio) in a solution maintained at a pH of 7.2 to 7.6 containing at least one stabilizing excipient. For long-term storage, the combined solution is lyophilized with one or more cryoprotectants, such as trehalose and mannitol.

### Example 2—Advanced PLGA Microsphere Preparation

### Materials

PLGA polymer (including blends of standard and end-capped PLGA)

Human insulin, Human C-peptide

Trehalose dihydrate, Human serum albumin, High-purity Polysorbate 20, L-Arginine, Zinc Chloride

Dichloromethane or Ethyl Acetate (pharmaceutical grade)

Aqueous solutions for pH control

### Method

Aqueous Phase (W1) Preparation and Pre-Stabilization: Dissolve insulin, C-peptide, and zinc chloride in the aqueous phase (W1). Add trehalose.

Organic Phase (O) Preparation: Dissolve a blend of 50:50 PLGA and 75:25 end-capped PLGA in ethyl acetate.

Primary Emulsion (W1/O) Formation: Emulsify W1 into O using a microfluidic device to create uniform droplets.

Secondary Emulsion & Solvent Removal: Transfer the primary emulsion to an external aqueous phase and evaporate the solvent.

Microsphere Collection and Washing for Burst Control: Collect the formed microspheres by centrifugation.

Wash with a pH 7.0 solution containing 0.1% Polysorbate 20 to remove surface-adsorbed protein.

Final Lyophilization: Lyophilize the washed microspheres to produce a final powder.

### Example 3—Optimized Lyophilized Formulation Preparation Materials

Human insulin, Human C-peptide, Trehalose dihydrate, Mannitol, L-Arginine hydrochloride, L-Histidine, L-Methionine, Ethylenediaminetetraacetic acid sodium salt, or EDTA disodium salt, Polysorbate 80 (high-purity, low-peroxide grade), Zinc chloride, components for pH control, Water for injection.

### Method

Solution Preparation: Prepare separate stock solutions of insulin and C-peptide.

In a main compounding vessel, combine excipients including zinc chloride, trehalose, mannitol in a 5:1 weight ratio, L-arginine, polysorbate 80, L-methionine, and EDTA in an aqueous solution.

Final Assembly: Add the insulin and C-peptide stocks to the main vessel to achieve final target concentrations and a pH from 7.2 to 7.6.

Sterile Filtration & Filling: The final solution is sterile filtered and filled into vials.

Lyophilization Cycle: Execute a controlled freeze-drying cycle optimized for the trehalose/mannitol ratio.

### Example 4—Particle Size Optimization for Controlled Release (Prophetic Example)

To achieve specific release durations, PLGA microspheres were engineered with defined particle size distributions. Microspheres sized 200-400 nm were optimized for 12-18 hour release, 400-1000 nm for 18-24 hour release, and 5-20 μm for 1-week depot release. Particle size was controlled using microfluidic emulsification and verified by dynamic light scattering (DLS) and scanning electron microscopy (SEM).

### Example 5—Comparative Stability Study (Prophetic Example)

Objective: To illustrate a potential difference in stability between the optimized insulin-C-peptide co-formulation and a formulation of insulin alone.

Proposed Methods: Formulations would be stored under accelerated conditions (25° C./60% RH). Stability would be assessed using SEC-HPLC, reverse-phase HPLC, and cell-based glucose uptake assays.

Expected Outcome: It is anticipated that the optimized co-formulation would demonstrate superior stability across all metrics compared to insulin alone.

While the preferred embodiments of the invention have been described above, it will be recognized and understood that various modifications may be made therein, and the appended claims are intended to cover all such modifications which may fall within the spirit and scope of the invention.

### REFERENCES

1. Abramson J, Adler J, Dunger J, Evans R, Green T, Pritzel A, et al. Accurate structure prediction of biomolecular interactions with AlphaFold 3. Nature. 2024; 630(8016):493-500.
2. Ahmad N, Wang W, Nair R, Kapila S. Relaxin induces matrix-metalloproteinases-9 and -13 via RXFP1: induction of MMP-9 involves the PI3K, ERK, Akt and PKC-zeta pathways. Mol Cell Endocrinol. 2012; 363(1-2):46-61.
3. Albarrán-Juárez J, Hitzel J, Snetkov X, Offermanns S, Wettschureck N. Piezol-mediated mechanotransduction in vascular remodeling and disease. Trends Cell Biol. 2023; 33(5):343-356.
4. Al-Qahtani F S, Alshaikh A A, Alfaifi S H. The Association Between Vitamin D Deficiency and the Level of Fasting C Peptide Among Patients With Uncontrolled Type 2 Diabetes Mellitus: A Retrospective Cohort Study. Cureus. 2024 Apr. 16(4):e58133.
5. Al-Trad B, Al-Batayneh K, El-Metwally S, Al-khateeb H, Al-zoubi M. The role of C-peptide in the pathogenesis of type 1 diabetes mellitus. J Diabetes Complications. 2015; 29(5):731-7.
6. Anagnostis P, Athyros V G, Tziomalos K, Karagiannis A, Mikhailidis D P. The pathogenetic role of cortisol in the metabolic syndrome: a hypothesis. J Clin Endocrinol Metab. 2009; 94(8):2692-701.
7. Anagnostis P, Katsiki N, Adamidou F, Athyros V G, Karagiannis A, et al. The role of cortisol in the metabolic syndrome: a review. Hormones (Athens). 2013; 12(2):173-90.
8. Bagher P, Valdivia H H, van Helden D F, Smith D W. PIEZO1 in mechanotransduction of vascular smooth muscle. J Physiol. 2018; 596(23):5723-5735.
9. Bathgate R A, Ivell R, Sanborn B M, Sherwood O D, Summers R J. International Union of Pharmacology LVII: recommendations for the nomenclature of receptors for relaxin family peptides. Pharmacol Rev. 2006; 58(1):7-31.
10. Beaulieu J M, Gainetdinov R R. The physiology, signaling, and pharmacology of dopamine receptors. Pharmacol Rev. 2011; 63(1):182-217.
11. Boels K. Human C-peptide is a ligand of the elastin-receptor-complex and therewith central to human vascular remodelling and disease in metabolic syndrome. Med Hypotheses. 2022; 169:110964.
12. Bogdanova A, Petrushanko I Y, Hernansanz-Austin P, Martinez-Ruiz A; “Oxygen Sensing” by Na,K-ATPase: These miraculous thiols. Frontiers in Physiology; 7(134); 1-21; (2016).
13. Brown M S, Goldstein J L. The SREBP pathway: regulation of cholesterol metabolism by proteolysis of a membrane-bound transcription factor. Cell. 1997; 89(3):331-40.
14. Brownlee M; The pathobiology of diabetic complications: A unified mechanism, Banting Lecture 2004; Diabetes; 54:1615-1625; (2004).
15. Cargnello M, Roux P P. Activation and function of the MAPKs and their substrates, the MAPK-activated protein kinases. Microbiol Mol Biol Rev. 2011; 75(1):50-83.
16. Charmandari E, Chrousos G P, Kino T. Glucocorticoid resistance. J Clin Endocrinol Metab. 2018 March; 103(3):856-67.
17. Chandler P D, Giovannucci E L, Scott J B, Bennett G G, Ng K, Chan A T, et al. Effects of Vitamin D Supplementation on C-peptide and 25-hydroxyvitamin D Concentrations at 3 and 6 Months. Sci Rep. 2015; 5:10411.
18. Chen Q, Gong C X. The role of SIRT1 in the regulation of Alzheimer's disease. Mol Neurobiol. 2019; 56(7):4949-61.
19. Chen R, Lyu T, Wang Y, Xu X, Zhang S, Zheng Y, et al. The role of C-peptide in diabetes and its complications: an updated review. Front Endocrinol (Lausanne). 2023; 14:1256093.
20. Chen R, Chen T, Li X, et al. SREBP2 in cancer progression and cholesterol regulation. Front Pharmacol. 2025; 16:1535691.
21. Chen Z P, Mitchelhill K I, Michell B J, Stapleton D, Rodriguez-Crespo I, Witters L A, et al. AMP-activated protein kinase phosphorylation of endothelial N O synthase. FEBS Lett. 1999; 443(3):285-9.
22. Chen Z, Zhang H, Liu Q, Chen J, et al. GPR146 facilitates blood pressure elevation and vascular remodeling via PIEZO1. Circ Res. 2025 Jul. 10. doi: 10.1161/CIRCRESAHA.125.326288. [Epub ahead of print]. ***ERROR IN PUBLICATION assuming Gas, not the correct Gai. This error was taken into account with inclusion into the patent***
23. Choi Y J, Kim Y H, Cho C H, Kim S H, Lee J E. Circulating Concentrations of C-Peptide and Colorectal Adenoma. Clin Nutr Res. 2014; 3(1):17-23.
24. Chou S H, Wand G S. The metabolic syndrome and functional hypercortisolism: a bidirectional association? Ann N Y Acad Sci. 2016; 1367(1):45-56.
25. Chrousos G P, Kino T. Glucocorticoid signaling and the glucocorticoid resistance syndrome. N Engl J Med. 2009 May 14; 360(19):2005-10.
26. Chrivia J C, Kwok R P, Lamb N, Hagiwara M, Montminy M R, Goodman R H. Phosphorylated CREB binds specifically to the nuclear protein CBP. Nature. 1993; 365(6449):855-9.
27. Clarke N E, Belyaev N D, Lambert D W, Turner A J; Epigenetic regulation of angiotensin-converting enzyme 2 (ACE2) by SIRT1 under conditions of cell energy stress; Clin Sci.; 126(7): 507-516; (2014).
28. Colombe A S, Pidoux G. Cardiac cAMP-PKA signaling compartmentalization. Cells. 2021; 10(4):922.
29. DeFronzo R A, Tripathy D, Schwenke D C, Banerji M, Bray G A, et al. The Ominous Octet: A Unifying Pathophysiology of Type 2 Diabetes. Diabetes. 2009; 58(4):773-95.
30. Ding Y, Wang L, Zhu Y, Wang Z, Song Z. Diabetes mellitus and idiopathic pulmonary fibrosis: a Mendelian randomization study. J Thorac Dis. 2024 Mar. 16(3):2144-50.
31. D'Onofrio N, Servillo L, Balestrieri L; SIRT1 and SIRT6 signaling pathways in cardiovascular disease protection; Antioxidants and Redox Signaling; 2018; 28(8); 711-732.
32. Dschietzig T, Bartsch C, Stangl V, Baumann G, Stangl K. RXFP1-inactive relaxin activates human glucocorticoid receptor: further investigations into the relaxin-GR pathway. Regul Pept. 2009; 154(1-3):77-84.
33. Dschietzig T, Bartsch C, Stangl V, Baumann G, Stangl K. Autoregulation of human relaxin-2 gene expression critically involves relaxin and glucocorticoid receptor binding to glucocorticoid response half-sites in the relaxin-2 promoter. J Mol Endocrinol. 2009; 42(4):315-28.
34. Du W, Ma X, Liang X, Zhang Y, Fu X, Wang N, et al. PIEZO1-mediated calcium influx is involved in the pathogenesis of pulmonary hypertension. Basic Res Cardiol. 2021; 116(1):16.
35. Figueiredo K A, Rossi G E, Lazarini C A, Casatti C A, Lopes-de-Souza S, et al. Relaxin interacts with the human glucocorticoid receptor. Peptides. 2006; 27(12):3243-8.
36. Forst T, Kunt T, Pohlmann T, Goitom K, Engelbach M, et al. Biological activity of C-peptide on the skin microcirculation in patients with type 1 diabetes. J Clin Invest. 1998; 101(10):2036-41.
37. Galicia-Garcia U, Benito-Vicente A, Jebari S, Larrea-Sebal A, Siddiqi H, et al. Pathophysiology of Type 2 Diabetes Mellitus. Int J Mol Sci. 2020; 21(17):6275.
38. Ganella D E, Ryan P J, Bathgate R A, Lawrence A J, Gundlach A L. Increased anxiety-like behaviour in rats following acute central administration of a relaxin-3 receptor (RXFP3) agonist. Behav Brain Res. 2012; 228(1):231-9.
39. Gilman A G. G proteins: transducers of receptor-generated signals. Annu Rev Biochem. 1987; 56:615-49.
40. Goeddel D V, Kleid D G, Bolivar F, Heyneker H L, Yansura D G, Crea R, et al. Expression in Escherichia coli of chemically synthesized genes for human insulin. Proc Natl Acad Sci USA. 1979; 76(1):106-10.
41. Halls M L, Bathgate R A, Summers R J. Relaxin family peptide receptors RXFP1 and RXFP2 modulate cAMP signaling by distinct mechanisms. Mol Pharmacol. 2006; 70(1):214-26.
42. Halls M L, van der Westhuizen E T, Bathgate R A, Summers R J. Relaxin family peptide receptors—former orphans reunite with their parent ligands. Br J Pharmacol. 2007; 151(4):427-41.
43. Han F, Liu W, Li C, Yang L, Chang C, et al. GPR146 Deficiency Protects against Hypercholesterolemia and Atherosclerosis. Cell. 2019; 179(6):1276-1288.e14.
44. Hills C E, Brunskill N J. Intracellular signalling by C-peptide. Exp Diabetes Res. 2008; 2008:635158.
45. Horton J D, Goldstein J L, Brown M S. SREBPs: activators of the complete program of cholesterol and fatty acid synthesis in the liver. J Clin Invest. 2002; 109(9):1125-31.
46. Hsu C-N, Chang C-H, Lin Y-S, Lin J-W, Caffrey J L. Association of Serum C-Peptide Concentrations with Cancer Mortality Risk in Pre-Diabetes or Undiagnosed Diabetes. PLoS One. 2013; 8(2):e55625
47. Hu X, Chen F, Jia L, Long A, Peng Y, Li X, Huang J, Wei X, Fang X, Gao Z, Zhang M, Liu X, Chen Y G, Wang Y, Zhang H, Wang Y. A gut-derived hormone regulates cholesterol metabolism. Cell. 2024 Mar. 28; 187(7):1685-1700.e18. doi: 10.1016/j.cell.2024.02.024.
48. James H, Gonsalves W I, Manjunatha S, Dasari S, Lanza I R, Klaus K A, et al. The Effect of Glucagon on Protein Catabolism During Insulin Deficiency: Exchange of Amino Acids Across Skeletal Muscle and the Splanchnic Bed. Diabetes. 2022 August; 71(8):1636-48.
49. Jeon T I, Osborne T F. SREBPs: metabolic integrators in physiology and metabolism. Trends Endocrinol Metab. 2012; 23(2):65-72.
50. Jarnvall H, Johansson J, Wahren J. Proinsulin C-peptide: a chaperone and an anchor. Biomol Concepts. 2014; 5(2):109-118. Biomol Concepts. 2014; 5(2):109-118.
51. Kahsai A W, Shah K S, Shim P J, et al. Allosteric activation of ERK MAPK by β-arrestin. Proc Natl Acad Sci USA. 2023; 120(42):e2303794120.
52. Kim J W, Kim Y J. Cholesin and GPR146 in modulating cholesterol biosynthesis. Pharmacology. 2024; 109(6):305-11.
53. Kim K, Han Y, Duan L, Chung K Y. Scaffolding of MAPK signaling by β-arrestins. Int J Mol Sci. 2022; 23(2):1000.
54. Kitamura T, Kimura K, Jung B D, Makondo K, Okamoto S, et al. Proinsulin C-peptide increases nitric oxide production by enhancing mitogen-activated protein-kinase-dependent transcription of endothelial nitric oxide synthase. Diabetologia. 2003; 46(12):1698-705.
55. Landreh M, Alvelius G, Wahren J, Jörnvall H, Johansson J. Insulin solubility transitions by pH-dependent interactions with proinsulin C-peptide. FEBS J. 2012; 279(23):4589-4597.
56. Lee M H, Kim B, Kim Y M, Choi S, Lee Y S. β-Arrestin-2-dependent activation of AMP-activated protein kinase is essential for adenosine A2A receptor-mediated inhibition of vascular smooth muscle cell migration. J Biol Chem. 2020; 295(10):3046-3058.
57. Li Y, Xu S, Mihaylova M M, Zheng B, Hou X, Jiang B, et al., AMPK phosphorylates and inhibits SREBP activity to attenuate hepatic steatosis and atherosclerosis. Cell Metab, 2011; 14(3), 361-71.
58. Li Y, Zhong Y, Gong W, Gao X, Qi H, Liu K, Qi J; C-peptide prevents SMAD3 binding to alpha promoters to inhibit collagen type 4 synthesis; Journal of Molecular Endocrinology; 2018; 61(1): 47-56.
59. Li Y C, Kong J, Wei M, Chen Z, Liu S Q, Li L. 1,25-Dihydroxyvitamin D(3) is a negative endocrine regulator of the renin-angiotensin system. J Clin Invest. 2004; 114(2):228-38.
60. Lindfors L, Sundström L, Fröderberg Roth L, Meuller J, Andersson S, Kihlberg J. Is GPR146 really the receptor for proinsulin C-peptide? Bioorg Med Chem Lett. 2020; 30(14):127278.
61. Liu J, Zhang J, Li H. SREBPs in lipid metabolism, insulin signaling, and beyond. World J Diabetes. 2018; 9(4):47-62.
62. Lopez D, BBA MCR. Transcriptional Regulation of Cholesterol and Steroid Metabolism by the SREBP Pathway. BBA—Molecular and Cell Biology of Lipids. 2021; 1866(4):158891.
63. Luo J, Yang H, Song B L. Mechanisms and regulation of cholesterol homeostasis. Nat Rev Mol Cell Biol. 2020; 21(4):225-45.
64. Liitjohann D, Stella J R. GPR146: a new player in cholesterol metabolism. Curr Opin Lipidol. 2023; 34(3):121-7.
65. Madison B B. SREBPs: The dream team of master regulators of lipid metabolism. Mol Metab. 2016; 5(3):153-5.
66. Mayr B, Montminy M. Transcriptional regulation by the phosphorylation-dependent factor CREB. Nat Rev Mol Cell Biol. 2001; 2(8):599-609.
67. McGowan B M, Stanley S A, Smith K L, Minnion J S, Donovan J, et al. Central relaxin-3 administration causes a profound anorexigenic effect in male rats. Endocrinology. 2005; 146(8):3295-300.
68. McGowan B M, Stanley S A, Smith K L, Minnion J S, Donovan J, et al. Effects of the neuropeptide relaxin-3 on food intake and the hypothalamic-pituitary-gonadal axis in the rat. Am J Physiol Endocrinol Metab. 2008; 295(2):E425-34.
69. McGowan B M, Stanley S A, Smith K L, White N E, Connolly M M, et al. Relaxin-3 stimulates the neuro-endocrine stress axis via corticotrophin-releasing hormone. J Endocrinol. 2014; 221(2):337-46.
70. Michaud D S, Wolpin B, Giovannucci E, Liu S, Cochrane B, Manson J E, Pollak M N, Ma J, Fuchs C S. Prediagnostic plasma C-peptide and pancreatic cancer risk in men and women. Cancer Epidemiol Biomarkers Prev. 2007; 16(10):2101-2109.
71. Nariai Y, Shimomura T, Togami K, Uesugi H, Nakae I, Ohta M, et al. Glucocorticoids Repress CREB-Binding Protein (CBP) Expression in A549 Cells. Am J Physiol Lung Cell Mol Physiol. 1997; 273(5):L1057-63
72. Nerelius C, Johansson J, Wahren J, Söderberg-Ahlm C. Proinsulin C-peptide affects the fibril formation of AP1-40 and Aβ1-42. Biochem Biophys Res Commun. 2010; 403(3-4):462-467.
73. Ozaki I, Zhang X, Ota T, Mizuta T, Eguchi Y, et al. MEK/ERK signaling is a critical regulator of SREBP-2-dependent lipid homeostasis. Cell Rep. 2016; 17(1):1-10.
74. Patel, V B, Zhong, J C, Grant M B, Oudit, G Y; Role of ACE2/Angiotensin 1-7 axis of the renin-angiotensin system in heart failure; Circ. Res.; 2016118(8): 1313-1326.
75. Peterson T R, Sengupta S, Harris T E, Carmack A E, Kang S A, Shimobayashi M, et al. mTOR complex 1 regulates lipin 1 localization to control the SREBP pathway. Cell, 2011; 146(3), 408-20.
76. Piao X, et al. G-protein-coupled receptor ADGRG1 drives a protective microglial state in Alzheimer's disease through MYC activation. Neuron. 2025; [epub ahead of print].
77. Pivonello R, Isidori A M, De Martino M C, Colao A. The role of cortisol in the pathogenesis of the metabolic syndrome. Best Pract Res Clin Endocrinol Metab. 2016; 30(3):331-44.
78. Pinger C W, Hawa M I, Clayton D, Heneghan C, Torkington A, O'Brien S, et al. C-Peptide replacement therapy in type 1 diabetes: are we in the trough of disillusionment? Mol BioSyst. 2017; 13(8):1432-7.
79. Pittas A G, Nelson J, Mitri J, Hillmann W, Garganta C, Nathan D M, et al. Plasma 25-hydroxyvitamin D and progression to diabetes in patients at risk for diabetes: an ancillary analysis in the Diabetes Prevention Program. Diabetes Care. 2012 March; 35(3):565-73.
80. Pollakova D, Careddu G, Dal Bello F, Querci F, Tanda M L, Zanin M E, et al. Muscular involvement in long-term type 1 diabetes: Does it represent an underestimated complication?. Nutrition. 2023 July-August; 111-112:112060.
81. Radhakrishnan A, Sun L P, Kwon H J, Brown M S, Goldstein J L. Direct binding of cholesterol to the SREBP-cleavage-activating protein in vitro. Proc Natl Acad Sci USA. 2004; 101(34):12462-7.
82. Ranade S S, Woo S H, Dubin A E, et al. Piezol, a mechanically activated ion channel, is required for vascular development in mice. Nature. 2015; 516(7529):123-127.
83. Rigler R, Pramanik A, Jonasson P, Kratz G, Jansson 0, et al. Specific binding of proinsulin C-peptide to human cell membranes. Proc Natl Acad Sci USA. 1999; 96(23):13318-23.
84. Ryan P J, Büchi M, R-K, Rosengren K J, Gundlach A L, Lawrence A J. Relaxin-3/RXFP3 system and its role in anxiety and depression. Cell Mol Life Sci. 2013; 70(24):4749-61.
85. She L, Yao H, He L, Li Y, Cao J. GPR146: an emerging therapeutic target for hypercholesterolemia and atherosclerosis. Acta Biochim Biophys Sin. 2020; 52(8):914-5.
86. Shen Y, Li M, Wang K, Qi G, Liu H, Wang W, Ji Y, Chang M, Deng C, Xu F, Shen M, Sun H. Diabetic muscular atrophy: molecular mechanisms and promising therapies. Front Endocrinol (Lausanne). 2022; 13:933357.
87. Shimano H. SREBPs: Physiology and pathophysiology of the SREBP family. FEBS J. 2021; 288(1), 31-39.
88. Shimano H, Sato R. SREBP-regulated lipid metabolism: convergent physiology-divergent pathophysiology. Nat Rev Endocrinol. 2017; 13(12):710-30.
89. Shimano H, Yahagi N, Amemiya-Kudo M, Hasty A H, Osuga J, Tamura Y, et al. Sterol regulatory element-binding protein-1 as a key transcription factor for nutritional induction of lipogenic enzyme genes. J Biol Chem. 1999; 274(50):35832-9.
90. Smith C M, Hosken I T, Sutton S W, Lawrence A J, Gundlach A L. Relaxin-3/RXFP3 signaling and neuroendocrine function—A perspective on extrinsic hypothalamic control. Front Endocrinol (Lausanne). 2013; 4:128.
91. Smith C M, Shen P J, Ban-G, Chua B E, G, et al. The relaxin-3/RXFP3 system in the mouse brain: Effects of receptor deficiency on anxiety- and depression-like behavior. Behav Brain Res. 2013; 242:128-37.
92. Smith T E, Whalen K L, Lefkowitz R J. Biased agonism: a new paradigm in G protein-coupled receptor signaling. Curr Opin Pharmacol. 2018; 41:7-15.
93. Spence D M, Patel H, Nolan B, Keyzers R, Libs P, White B, et al. A C-peptide complex with albumin and Zn2+ increases measurable GLUT1 levels in membranes of human red blood cells. Sci Rep. 2020; 10(1):17493.
94. Steiner D F, Oyer P E. The biosynthesis of insulin and a probable precursor of insulin by a human islet cell adenoma. Proc Natl Acad Sci USA. 1967; 57(2):473-80.
95. Steiner D F. The proinsulin C-peptide—a multi-role peptide. Exp Diabesity Res. 2004; 5(1):7-14.
96. Stewart T A, Yapa KTDS, Monteith G R; Altered calcium signalling in cells; Biochimica et Biophysica Acta—Biomembranes; 1848(10): 2502-2511; (2015).
97. Tanaka M, Iijima N, Miyamoto Y, Fukusumi S, Itoh Y, et al. Characterization of a novel human neuropeptide, relaxin-3, and its receptor, GPCR135. J Biol Chem. 2005; 280(10):8939-45.
98. Tanaka M, Iijima N, Miyamoto Y, Fukusumi S, Itoh Y, et al. Central administration of relaxin-3 induces c-fos expression in the rat brain. Regul Pept. 2005; 126(1-2):55-60.
99. The Diabetes Control and Complications Trial Research Group. The effect of intensive treatment of diabetes on the development and progression of long-term complications in insulin-dependent diabetes mellitus. N Engl J Med. 1993; 329(14):977-86.
100. UniProt Consortium. GPR146-Homo sapiens. UniProtKB Q96CH1. https://www.uniprot.org/uniprotkb/Q96CH1/entry. Accessed Sep. 23, 2025.
101. van der Sluis B, Zhang B, Kuivenhoven J A. GPR146: an emerging therapeutic target for hypercholesterolemia and atherosclerosis. Curr Opin Lipidol. 2022; 33(3):151-7.
102. Vassar R. BACE1: The β-Secretase Enzyme in Alzheimer's Disease. J Mol Neurosci. 2009; 39(3):374-82.
103. Velloso L A, Folli F, Saad M J. The role of C-peptide in the signal transduction of insulin. Diabetologia. 2002; 45(11):1457-69.
104. Venugopal S K, Mowery M L, Jialal I. Biochemistry, C Peptide. In: StatPearls [Internet]. Treasure Island (F L): StatPearls Publishing; 2023 [Updated 2023 Aug. 1]. Available from: https://www.ncbi.nlm.nih.gov/books/NBK526026/
105. Vergnes L, Chin R G, de Aguiar Vallim T Q, Fong L G, Osborne T F, et al. SREBP-2 is required for fatty acid and cholesterol synthesis in the developing mouse liver. J Biol Chem. 2016; 291(11):5779-90.
106. Wahren J, Kallas A, Sima A A. The clinical potential of C-peptide replacement in Type 1 diabetes. Diabetes. 2012; 61(4):761-72.
107. Wang, S, Wei, W, et al.; The Role of Insulin C-Peptide in the Coevolution Analyses of the Insulin Signaling Pathway: A Hint for Its Functions; PLOS ONE; (7)12; 1-9; (2012).
108. Wang Y, Viscarra J, Kim S J, Sul H S. Transcriptional regulation of hepatic lipogenesis. Nat Rev Mol Cell Biol. 2015; 16(11):678-89.
109. Watanabe Y, Iida Y, Yamada K, Miyamoto Y, Fukusumi S, et al. Relaxin-3/RXFP3 system regulates the hypothalamic-pituitary-adrenal axis in rats. J Mol Neurosci. 2011; 43(2):169-75.
110. Watanabe Y, Miyamoto Y, Matsuda F, Iijima N, Fukusumi S, et al. Centrally administered relaxin-3 induces Fos expression in the osmosensitive areas in rat brain and facilitates water intake. Peptides. 2010; 31(6):1051-7.
111. Whirledge S, Cidlowski J A. Glucocorticoids, stress, and fertility. Minerva Endocrinol. 2010; 35(2):109-25.
112. Wu S, Cao L, Tang J, Wang Y, Zhang X, Zhou H. The critical role of Piezol channel in cardiovascular diseases. Cell Signal. 2020; 75:109761.
113. Xu B, Tang Z, Li Z, Liu Z, Wu Z. Glucocorticoids inhibit CREB-binding protein (CBP) expression through a mechanism involving microRNA-140 in skeletal muscle cells. J Biol Chem. 2012; 287(23):19223-30.
114. Yosten G L C, Kolar G R. The Physiology of Proinsulin C-Peptide: Unanswered Questions and a Proposed Model. Physiology (Bethesda). 2015; 30(5):327-32.
115. Yosten G L C, Kolar G R, Redlinger L J, Samson W K. Evidence for an interaction between proinsulin C-peptide and GPR146. J Endocrinol. 2013; 218(2):B1-8.
116. Yu H, Rimbert A, Palmer A E, Toyohara T, Xia Y, Xia F, Ferreira L M R, et al. GPR146 deficiency protects against hypercholesterolemia and atherosclerosis. Cell. 2019; 179(6):1276-88.
117. Zahn U. Forty years of C-peptide. Diabetologia. 2007; 50(10):2013-5.
118. Zhang B, van der Sluis B, Kuivenhoven J A. G protein-coupled receptor 146: a novel regulator of lipoprotein metabolism and target to reduce cholesterol. University of Groningen; 2023.

1. A pharmaceutical composition for restoring physiological hormone coordination, comprising: (a) human insulin; and (b) human C-peptide; wherein the C-peptide and insulin are co-formulated in a single aqueous solution at a molar ratio and concentration sufficient to restore a physiological signaling dynamic at a GPR146 receptor, wherein an insulin-mediated signal is subsequently terminated by C-peptide-induced receptor internalization.

2. The pharmaceutical composition of claim 1 wherein the human insulin comprises an A chain having the amino acid sequence as set forth in SEQ ID No: 1 and a B chain having the amino acid sequence as set forth in SEQ ID No: 2.

3. The pharmaceutical composition of claim 1 wherein the C-peptide comprises the amino acid sequence as set forth in SEQ ID No: 3.

4. The pharmaceutical composition of claim 1, wherein the C-peptide to insulin molar ratio is between 0.8:1 and 1.2:1.

5. The pharmaceutical composition of claim 1, wherein the composition is formulated as a lyophilized powder.

6. The pharmaceutical composition of claim 1 further comprising a cryoprotectant.

7. The pharmaceutical composition of claim 6 further comprising a bulking agent at a weight ratio of 1:3 to 1:10 to the cryoprotectant.

8. The pharmaceutical composition of claim 7, wherein the cryoprotectant is trehalose and the bulking agent is mannitol, and the weight ratio is approximately 5:1.

9. The pharmaceutical composition of claim 1 further comprising one or more excipients selected from the group consisting of: a histidine buffer; a zinc compound; a sacrificial antioxidant; a chelating agent; and a surfactant, formulated to provide a pH of 7.2 to 7.6 upon reconstitution.

10. The pharmaceutical composition of claim 9, wherein the one or more excipients comprises a sacrificial antioxidant and the sacrificial antioxidant is L-methionine.

11. The pharmaceutical composition of claim 9 wherein the one or more excipients comprises a chelating agent and the chelating agent is EDTA sodium salt.

12. A sustained-release pharmaceutical composition comprising: (a) a biodegradable polymer matrix comprising a blend of polymers configured to control an initial burst release; (b) human insulin; and (c) human C-peptide, co-encapsulated within said polymer matrix to provide for simultaneous release.

13. The sustained release pharmaceutical composition of claim 12 wherein the human insulin comprises an A chain having the amino acid sequence as set forth in SEQ ID No: 1 and a B chain having the amino acid sequence as set forth in SEQ ID No: 2.

14. The sustained release pharmaceutical composition of claim 12 wherein the C-peptide consists of the amino acid sequence as set forth in SEQ ID No: 3.

15. The sustained-release composition of claim 12, wherein the biodegradable polymer is poly(lactic-co-glycolic acid) (PLGA) formed into microspheres with a mean particle diameter selected to provide a release duration of between 12 hours and 8 days.

16. The sustained-release composition of claim 15, wherein the microspheres are produced by a microfluidic process that includes a pre-stabilization step of complexing the insulin and C-peptide with zinc.

17. A method for preparing the pharmaceutical composition of claim 1, comprising: (a) combining aqueous solutions of insulin and C-peptide to achieve the desired molar ratio; (b) adding a stabilizing excipient; and (c) adjusting the pH to between 7.2 and 7.6 using a histidine buffer.


SEQUENCE LISTING (3 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 31 aa, Homo sapiens
EAEDLQVGQVELGGGPGAGSLQPLALEGSLQ